(19) United States
(12) Patent Application Publication
(10) Pub. No.: US 2022/0167920 A1
(43) Pub. Date: Jun. 2, 2022

(54) METHODS AND APPARATUS FOR RENAL NEUROMODULATION

(71) Applicant: PHILIPS IMAGE GUIDED THERAPY CORPORATION, SAN DIEGO, CA (US)

(72) Inventor: Marja Pauliina MARGOLIS, CORAL GABLES, FL (US)

(21) Appl. No.: 17/671,933

(22) Filed: Feb. 15, 2022

Related U.S. Application Data
(63) Continuation of application No. 13/458,856, filed on Apr. 27, 2012.

(51) Int. Cl. A61B 5/00; A61B 5/01; A61B 5/145; A61B 18/14

(52) U.S. Cl. CPC A61B 5/6858; A61B 5/01; A61B 8/12; A61B 18/1492; A61B 5/145

(57) ABSTRACT

A thermal neuromodulation apparatus, system, and methods for the ablative and non-ablative application of thermal energy to the renal nerves of a patient are disclosed. The thermal neuromodulation apparatus includes an elongated, hollow body configured to traverse the tortuous intravascular pathways of the renal vasculature and includes an expandable structure bearing electrodes and configured to selectively apply thermal energy via electric fields to the renal nerves through a vessel wall. The thermal neuromodulation apparatus may also include sensors and an imaging apparatus to obtain data from the treatment area before, during, and after neuromodulation to monitor and/or control the neuromodulation process.

### CROSS-REFERENCE TO PRIOR APPLICATIONS

This is a Continuation of application Ser. No. 13/458,856, filed Apr. 27, 2012 which application is hereby incorporated by reference herein.

### TECHNICAL FIELD

Embodiments of the present disclosure relate generally to the field of medical devices and, more particularly, to an apparatus, systems, and methods for achieving intravascular neuromodulation.

### BACKGROUND

Hypertension and its associated conditions, chronic heart failure (CHF) and chronic renal failure (CRF), constitute a significant and growing global health concern. Current therapies for these conditions span the gamut covering non-pharmacological, pharmacological, surgical, and implanted device-based approaches. Despite the vast array of therapeutic options, the control of blood pressure and the efforts to prevent the progression of heart failure and chronic kidney disease remain unsatisfactory.

Blood pressure is controlled by a complex interaction of electrical, mechanical, and hormonal forces in the body. The main electrical component of blood pressure control is the sympathetic nervous system (SNS), a part of the body's autonomic nervous system, which operates without conscious control. The sympathetic nervous system connects the brain, the heart, the kidneys, and the peripheral blood vessels, each of which plays an important role in the regulation of the body's blood pressure. The brain plays primarily an electrical role, processing inputs and sending signals to the rest of the SNS. The heart plays a largely mechanical role, raising blood pressure by beating faster and harder, and lowering blood pressure by beating slower and less forcefully. The blood vessels also play a mechanical role, influencing blood pressure by either dilating (to lower blood pressure) or constricting (to raise blood pressure).

The kidneys play a central electrical, mechanical and hormonal role in the control of blood pressure. The kidneys affect blood pressure by signaling the need for increased or lowered pressure through the SNS (electrical), by filtering blood and controlling the amount of fluid in the body (mechanical), and by releasing key hormones that influence the activities of the heart and blood vessels to maintain cardiovascular homeostasis (hormonal). The kidneys send and receive electrical signals from the SNS and thereby affect the other organs related to blood pressure control. They receive SNS signals primarily from the brain, which partially control the mechanical and hormonal functions of the kidneys. At the same time, the kidneys also send signals to the rest of the SNS, which can boost the level of sympathetic activation of all the other organs in the system, effectively amplifying electrical signals in the system and the corresponding blood pressure effects. From the mechanical perspective, the kidneys are responsible for controlling the amount of water and sodium in the blood, directly affecting the amount of fluid within the circulatory system. If the kidneys allow the body to retain too much fluid, the added fluid volume raises blood pressure. Lastly, the kidneys produce blood pressure regulating hormones including renin, a hormone that activates a cascade of events through the renin-angiotensin-aldosterone system (RAAS). This cascade, which includes vasoconstriction, elevated heart rate, and fluid retention, can be triggered by sympathetic stimulation. The RAAS operates normally in non-hypertensive patients but can become overactive among hypertensive patients. The kidney also produces cytokines and other neurohormones in response to elevated sympathetic activation that can be toxic to other tissues, particularly the blood vessels, heart, and kidney. As such, overactive sympathetic stimulation of the kidneys may be responsible for much of the organ damage caused by chronic high blood pressure.

Thus, overactive sympathetic stimulation of the kidneys plays a significant role in the progression of hypertension, CHF, CRF, and other cardio-renal diseases. Heart failure and hypertensive conditions often result in abnormally high sympathetic activation of the kidneys, creating a vicious cycle of cardiovascular injury. An increase in renal sympathetic nerve activity leads to the decreased removal of water and sodium from the body, as well as increased secretion of renin, which leads to vasoconstriction of blood vessels supplying the kidneys. Vasoconstriction of the renal vasculature causes decreased renal blood flow, which causes the kidneys to send afferent SNS signals to the brain, triggering peripheral vasoconstriction and increasing a patient's hypertension. Reduction of sympathetic renal nerve activity, e.g., via renal neuromodulation or denervation of the renal nerve plexus, may reverse these processes.

Efforts to control the consequences of renal sympathetic activity have included the administration of medications such as centrally acting sympatholytic drugs, angiotensin converting enzyme inhibitors and receptor blockers (intended to block the RAAS), diuretics (intended to counter the renal sympathetic mediated retention of sodium and water), and beta-blockers (intended to reduce renin release). The current pharmacological strategies have significant limitations, including limited efficacy, compliance issues, and side effects.

While the existing treatments have been generally adequate for their intended purposes, they have not been entirely satisfactory in all respects. The catheters, systems, and associated methods of the present disclosure overcome one or more of the shortcomings of the prior art.

### SUMMARY

In one exemplary embodiment, the present disclosure describes an apparatus for intravascular thermal neuromodulation, comprising an elongate, hollow body, and expandable structure, and at least one electrode. The elongate, hollow body includes a proximal portion and a distal portion including a distal tip. The body is configured to have an unexpanded condition wherein the distal portion and the distal tip are in contact with each other, and an expanded condition wherein the distal portion and the distal tip are spaced apart from each other. The expandable structure is configured to have an expanded condition and an unexpanded condition, and the expandable structure is disposed in an unexpanded condition within the distal portion and proximal to the distal tip. The expandable structure includes at least one support arm. The at least one electrode is positioned on the at least one support arm of the expandable structure.

In some instances, the expandable structure is configured for placement within a vessel lumen such that the at least one support arm contacts a vessel luminal wall when the expandable structure is in an expanded condition.

In another exemplary embodiment, the present disclosure describes an apparatus for intravascular thermal neuromodulation of the sympathetic renal nerve plexus comprising an elongate hollow body, an expandable structure, at least one electrode, and at least one sensor. The elongate, hollow body includes a proximal portion and a distal portion including a distal tip. The body is configured to have an unexpanded condition wherein the distal portion and the distal tip are in contact with each other, and an expanded condition wherein the distal portion and the distal tip are spaced apart from each other. The expandable structure is configured to have an expanded condition and an unexpanded condition. The expandable structure includes at least one support arm. The at least one electrode is positioned on the at least one support arm of the expandable structure, and the at least one sensor positioned on the expandable structure.

In another exemplary embodiment, the present disclosure describes an apparatus for intravascular thermal neuromodulation of the renal nerves overlying a renal artery, comprising an elongate, hollow body, an expandable structure, at least one electrode, and an imaging apparatus disposed on the body. The elongate, hollow body includes a proximal portion and a distal portion including a distal tip. The expandable structure includes at least one support arm. The at least one electrode is positioned on the at least one support arm of the expandable structure.

In another exemplary embodiment, the present disclosure describes a method for thermal modulation of nerves overlying a vessel, comprising positioning a thermal neuromodulation apparatus including an imaging apparatus and an expandable structure carrying at least one electrode within a lumen of the vessel, imaging a luminal wall of the vessel to obtain image data reflecting structural characteristics and a circumferential wall thickness of the lumen, positioning the thermal neuromodulation apparatus in an optimal intravascular location based on the image data, expanding the expandable structure to enable the at least one electrode to contact the luminal wall proximate the nerves, directing thermal energy from the at least one electrode through the luminal wall to the nerves, and imaging the luminal wall of the vessel and the nerves to obtain image data reflective of the extent of tissue damage.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 is a block diagram illustrating the pathophysiologic connection between the sympathetic nervous system, the brain, the peripheral vasculature, and the kidneys.

FIG. 2 is a schematic diagram illustrating the thermal basket catheter in an expanded condition according to one embodiment of the present disclosure positioned within the renal anatomy.

FIG. 3 is a schematic diagram illustrating a cross-sectional view of a segment of a renal artery.

FIG. 4a is a schematic diagram illustrating a perspective view of a portion of the renal nerve plexus overlying a segment of a renal artery.

FIG. 4b is a schematic diagram illustrating a perspective view of a portion of the renal nerve plexus overlying a segment of an atherosclerotic renal artery.

FIG. 4c is a schematic diagram illustrating a perspective view of a portion of the renal nerve plexus overlying a segment of a renal artery.

FIG. 5 is a schematic illustration of a thermal neuromodulation system including a thermal basket catheter according to one embodiment of the present disclosure.

FIG. 6a is an illustration of a side view of a portion of the thermal basket catheter in an unexpanded condition according to one embodiment of the present disclosure.

FIG. 6b is an illustration of a side view of a portion of the thermal basket catheter in an expanded condition according to one embodiment of the present disclosure.

FIG. 7 is an illustration of a partially cross-sectional side view of a portion of the thermal basket catheter in an unexpanded condition according to one embodiment of the present disclosure.

FIG. 8 is an illustration of a transverse cross-sectional view of the body of the thermal basket catheter as taken along the lines 8-8 of FIG. 7 according to one embodiment of the present disclosure.

FIG. 9 is an illustration of a cross-sectional side view of the expandable structure in a non-deployed and unexpanded condition according to one embodiment of the present disclosure.

FIG. 10 is an illustration of a cross-sectional view of a portion of the thermal basket catheter in an unexpanded condition according to one embodiment of the present disclosure.

FIG. 11 is an illustration of a perspective view of a portion of the thermal basket catheter in an unexpanded condition according to one embodiment of the present disclosure.

FIG. 12 is an illustration of a perspective view of a portion of the thermal basket catheter in an expanded condition according to one embodiment of the present disclosure.

FIG. 13 is an illustration of a perspective view of the expandable structure in an expanded condition according to one embodiment of the present disclosure.

FIG. 14 is an illustration of a plan view of the expandable structure in an expanded condition according to one embodiment of the present disclosure.

FIGS. 15a and 15b provide a schematic flowchart illustrating methods of delivering and controlling the thermal neuromodulation to renal vessels.

FIG. 16 is an illustration of a partially cross-sectional perspective view of a portion of the thermal basket catheter positioned within a vessel according to one embodiment of the present disclosure.

FIG. 17 is an illustration of a partially cross-sectional perspective view of a portion of a thermal basket catheter in an expanded condition positioned within a vessel according to one embodiment of the present disclosure.

FIG. 18a is an illustration of a partially cross-sectional perspective view of a portion of a thermal basket catheter in a partially expanded condition positioned within a vessel according to one embodiment of the present disclosure.

FIG. 18b is an illustration of a partially cross-sectional perspective view of a portion of the thermal basket catheter pictured in FIG. 18a in an expanded condition positioned within a vessel according to one embodiment of the present disclosure.

FIG. 19 is an illustration of a partially cross-sectional perspective view of a portion of a thermal basket catheter in an expanded condition positioned within a vessel according to one embodiment of the present disclosure.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. In addition, dimensions provided herein are for specific examples and it is contemplated that different sizes, dimensions, and/or ratios may be utilized to implement the concepts of the present disclosure. For the sake of brevity, however, the numerous iterations of these combinations will not be described separately. For simplicity, in some instances the same reference numbers are used throughout the drawings to refer to the same or like parts.

The present disclosure relates generally to an apparatus, systems, and methods of using thermal energy neuromodulation for the treatment of various cardiovascular diseases, including, by way of non-limiting example, hypertension, chronic heart failure, and/or chronic renal failure. In some instances, embodiments of the present disclosure are configured to deliver thermal energy to the renal nerve plexus to decrease renal sympathetic activity. Renal sympathetic activity may worsen symptoms of hypertension, heart failure, and/or chronic renal failure. In particular, hypertension has been linked to increased sympathetic nervous system activity stimulated through any of four mechanisms, namely (1) increased vascular resistance, (2) increased cardiac rate, stroke volume and output, (3) vascular muscle defects, and/or (4) sodium retention and renin release by the kidney. As to this fourth mechanism in particular, stimulation of the renal sympathetic nervous system can affect renal function and maintenance of homeostasis. For example, an increase in efferent renal sympathetic nerve activity may cause increased renal vascular resistance, renin release, and sodium retention, all of which exacerbate hypertension.

Thermal neuromodulation by either intravascular heating or cooling may decrease renal sympathetic activity by disabling the efferent and/or afferent sympathetic nerve fibers that surround the renal arteries and innervate the kidneys through renal denervation, which involves selectively disabling renal nerves within the sympathetic nervous system (SNS) to create at least a partial conduction block within the SNS. Thermal neuromodulation is due at least in part to the thermally-induced alterations of the neural structures themselves. Additionally or alternatively, the thermal neuromodulation may be due at least in part to the thermally-induced alteration of vascular structures, e.g. arteries, arterioles, capillaries, and/or veins, which perfuse the neural fibers surrounding the target area. Additionally or alternatively, the thermal neuromodulation may be due at least in part to the electroporation of the target neural fibers.

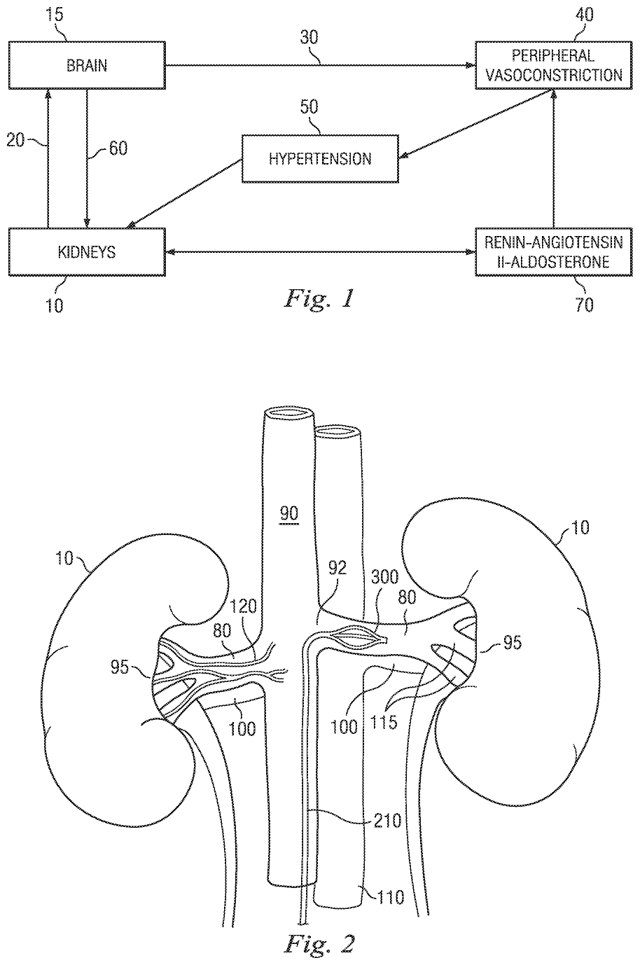

FIG. 1 illustrates the role of the kidneys 10 and renal nerve activity in the progression of hypertension. Several forms of renal injury or stress may induce activation of the renal afferent (from the kidney 10 to the brain 15 or the other kidney) signals 20. For example, renal ischemia, a reduction in stroke volume or renal blood flow, or an abundance of adenosine enzyme may trigger activation of renal afferent nerve activity 20. Increased renal afferent nerve activity 20 results in increased systemic sympathetic activation 30 and peripheral vasoconstriction (narrowing) 40 of blood vessels. Increased vasoconstriction results in increased resistance of blood vessels, which results in hypertension 50. Increased renal efferent (from the brain 15 to the kidney 10) nerve activity 60 results in further increased afferent renal nerve activity 20 and activation of the RAAS cascade 70, inducing increased secretion of renin, sodium retention, fluid retention, and reduced renal blood flow through vasoconstriction. The RAAS cascade 70 also contributes to systemic vasoconstriction of blood vessels 40, thereby exacerbating hypertension 50. In addition, hypertension 50 often leads to vasoconstriction and atherosclerotic narrowing of blood vessels supplying the kidneys 10, which causes renal hypoperfusion and triggers increased renal afferent nerve activity 20. In combination this cycle of factors results in fluid retention and increased workload on the heart, thus contributing to the further cardiovascular and cardio-renal deterioration of the patient. Therefore, FIG. 1 suggests how modulation of afferent and efferent sympathetic renal nerve activity may benefit patients with cardiovascular and cardio-renal diseases, including hypertension.

Renal denervation, which affects both the electrical signals going into the kidneys (efferent sympathetic activity 60) and the electrical signals emanating from them (afferent sympathetic activity 20), has the potential to impact the mechanical and hormonal activities of the kidneys 10 themselves, as well as the electrical activation of the rest of the SNS. Blocking efferent sympathetic activity 60 to the kidney may alleviate hypertension 50 and related cardiovascular diseases by reversing fluid and salt retention (augmenting natriuresis and diuresis), thereby lowering the fluid volume and mechanical load on the heart, and reducing inappropriate renin release, thereby halting the deleterious hormonal RAAS cascade 70 before it starts.

By blocking afferent sympathetic activity 20 from the kidney 10 to the brain 15, renal denervation may lower the level of activation of the whole SNS. Thus, renal denervation may also decrease the electrical stimulation of other members of the sympathetic nervous system, such as the heart and blood vessels, thereby causing additional anti-hypertensive effects. In addition, blocking renal nerves may also have beneficial effects on organs damaged by chronic sympathetic over-activity, because it may lower the level of cytokines and hormones that may be harmful to the blood vessels, kidney, and heart.

Furthermore, because renal denervation reduces overactive SNS activity, it may be valuable in the treatment of several other medical conditions related to hypertension. These conditions, which are characterized by increased SNS activity, include left ventricular hypertrophy, chronic renal disease, chronic heart failure, insulin resistance (diabetes and metabolic syndrome), cardio-renal syndrome, osteoporosis, and sudden cardiac death. For example, other benefits of renal denervation may theoretically include: reduction of insulin resistance, reduction of central sleep apnea, improvements in perfusion to exercising muscle in heart failure, reduction of left ventricular hypertrophy, reduction of ventricular rates in patients with atrial fibrillation, abrogation of lethal arrhythmias, and slowing of the deterioration of renal function in chronic kidney disease. Moreover, chronic elevation of renal sympathetic tone in various disease states that exist with or without hypertension may play a role in the development of overt renal failure and end-stage renal disease. Because the reduction of afferent renal sympathetic signals contributes to the reduction of systemic sympathetic stimulation, renal denervation may also benefit other organs innervated by sympathetic nerves. Thus, renal denervation may also alleviate various medical conditions, even those not directly associated with hypertension.

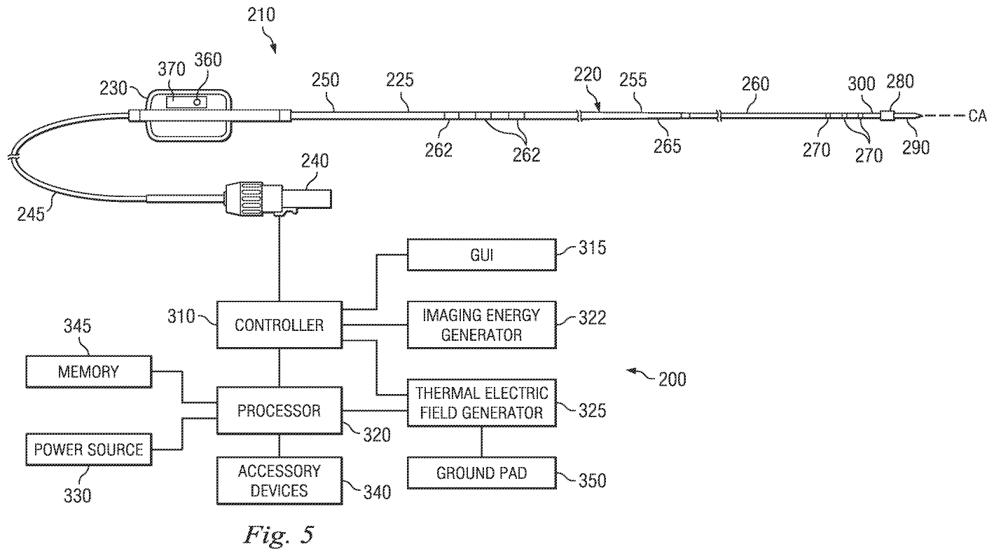

FIG. 2 illustrates a portion of a thermal basket catheter 210 in an expanded condition positioned within the human renal anatomy. The human renal anatomy includes kidneys 10 that are supplied with oxygenated blood by right and left renal arteries 80, which branch off an abdominal aorta 90 at the renal ostia 92 to enter the hilum 95 of the kidney 10. The abdominal aorta 90 connects the renal arteries 80 to the heart (not shown). Deoxygenated blood flows from the kidneys 10 to the heart via renal veins 100 and an inferior vena cava 110. Specifically, the thermal basket catheter 210 is shown extending through the abdominal aorta and into the left renal artery 80. In alternate embodiments, the thermal basket catheter may be sized and configured to travel through the inferior renal vessels 115 as well. The thermal basket catheter 210 will be described in more detail below with respect to FIGS. 5-18b.

Left (not shown) and right renal plexi or nerves 120 surround the left and right renal arteries 80, respectively. Anatomically, the renal nerve 120 forms one or more plexi within the adventitial tissue surrounding the renal artery 80. For the purpose of this disclosure, the renal nerve is defined as any individual nerve or plexus of nerves and ganglia that conducts a nerve signal to and/or from the kidney 10 and is anatomically located on the surface of the renal artery 80, parts of the abdominal aorta 90 where the renal artery 80 branches off the aorta 90, and/or on inferior branches of the renal artery 80. Nerve fibers contributing to the plexi 120 arise from the celiac ganglion, the lowest splanchnic nerve, the corticorenal ganglion, and the aortic plexus. The renal nerves 120 extend in intimate association with the respective renal arteries into the substance of the respective kidneys 10. The nerves are distributed with branches of the renal artery to vessels of the kidney 10, the glomeruli, and the tubules. Each renal nerve 120 generally enters each respective kidney 10 in the area of the hilum 95 of the kidney, but may enter in any location where a renal artery 80 or branch of the renal artery enters the kidney.

Proper renal function is essential to maintenance of cardiovascular homeostasis so as to avoid hypertensive conditions. Excretion of sodium is key to maintaining appropriate extracellular fluid volume and blood volume, and ultimately controlling the effects of these volumes on arterial pressure. Under steady-state conditions, arterial pressure rises to that pressure level which results in a balance between urinary output and water and sodium intake. If abnormal kidney function causes excessive renal sodium and water retention, as occurs with sympathetic overstimulation of the kidneys through the renal nerves 120, arterial pressure will increase to a level to maintain sodium output equal to intake. In hypertensive patients, the balance between sodium intake and output is achieved at the expense of an elevated arterial pressure in part as a result of the sympathetic stimulation of the kidneys through the renal nerves 120. Thermal neuromodulation of the renal nerves 120 may help alleviate the symptoms and sequelae of hypertension by blocking or suppressing the efferent and afferent sympathetic activity of the kidneys 10.

FIG. 3 illustrates a segment of the renal artery 80 in greater detail, showing various intraluminal characteristics and intra-to-extraluminal distances that may be present within a single vessel. In particular, the renal artery 80 includes a lumen 135 that extends lengthwise through the renal artery along a longitudinal axis LA. The lumen 135 is a tube-like passage that allows the flow of oxygenated blood from the abdominal aorta to the kidney. The sympathetic renal nerves 120 extend generally within the adventitia (not shown) surrounding the renal artery 80, and include both the efferent (conducting away from the central nervous system) and afferent (conducting toward the central nervous system) renal nerves.

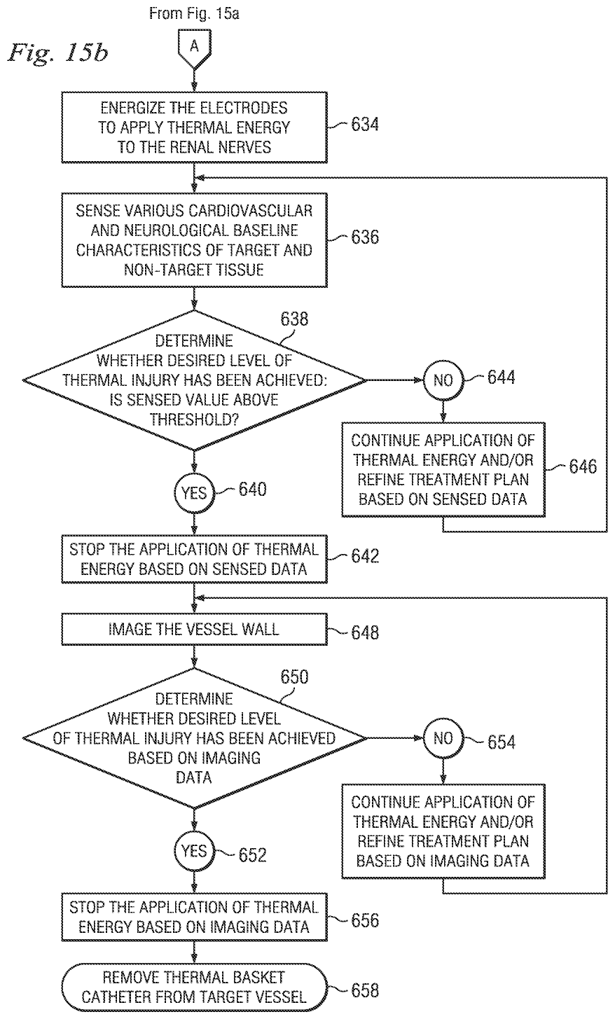

The renal artery 80 includes a first portion 141 having a generally healthy luminal diameter D1 and an intra-to-extraluminal distance D2, a second portion 142 having a narrowed and irregular lumen and an enlarged intra-to-extraluminal distance D3 due to atherosclerotic changes in the form of plaques 160, 170, and a third portion 143 having a narrowed lumen and an enlarged intra-to-extraluminal distance D2′ due to a thickened arterial wall 150. Thus, the intraluminal contour of a vessel, for example, the renal artery 80, may be greatly varied along the length of the vessel. Variable intra-to-extraluminal distances along the length of the vessel may affect the treatment protocols for implementing thermal neuromodulation at different portions of the vessel at least because the amount of thermal energy necessary to travel the intra-to-extraluminal distance to affect neural tissue surrounding the vessel varies with varying intra-to-extraluminal distances. As described further below in relation to FIG. 15, the thermal basket catheters disclosed herein may aid in determining appropriate and effective treatment protocols by pre-treatment, in-treatment, and post-treatment imaging and sensing of various characteristics.

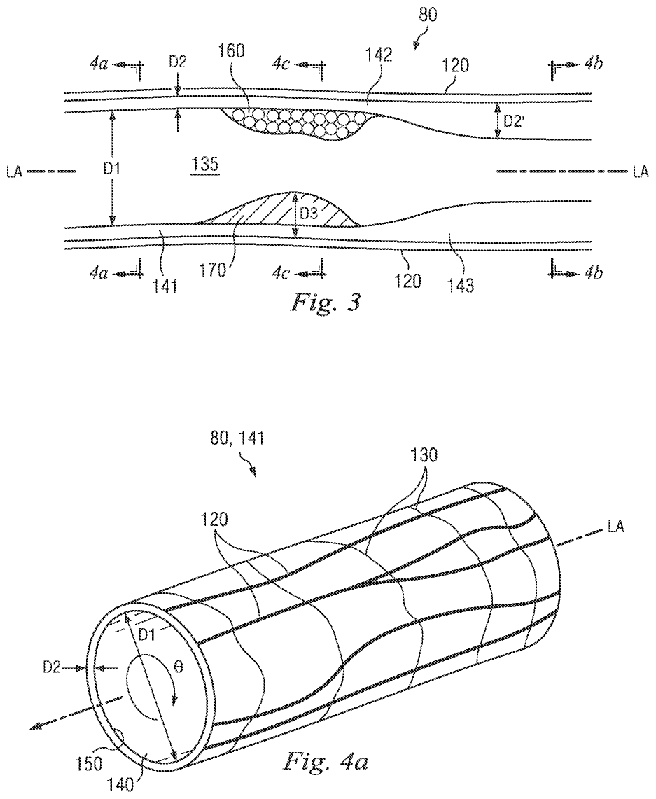

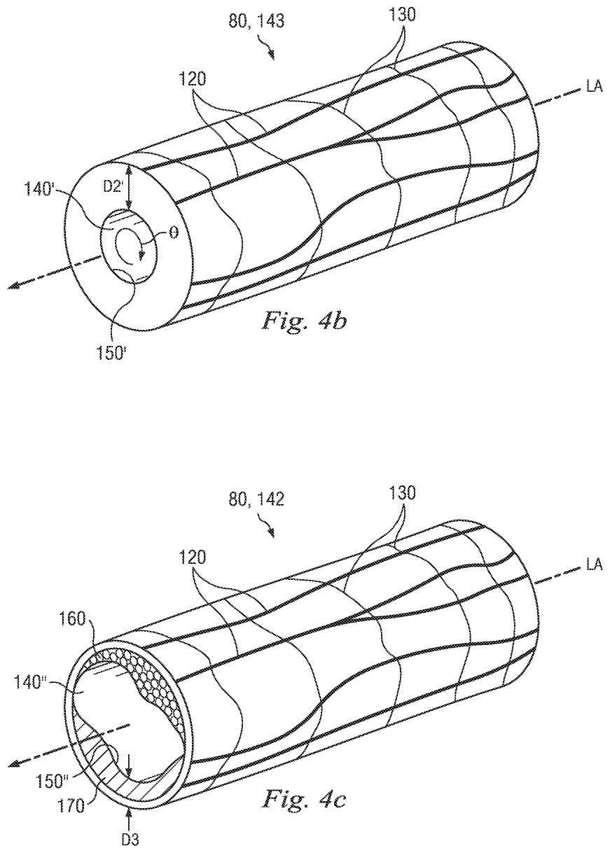

FIGS. 4a, 4b, and 4c illustrate the portions 141, 143, 142, respectively, of the renal artery 80 in perspective view, showing the sympathetic renal nerves 120 that line the renal artery 80. FIG. 4a illustrates the portion 141 of the renal artery 80 including the renal nerves 120, which are shown schematically as a branching network attached to the external surface of the renal artery 80. The renal nerves 120 extend generally lengthwise along the longitudinal axis LA of renal artery 80. In the case of hypertension, the sympathetic nerves that run from the spinal cord to the kidneys 10 signal the body to produce norepinephrine, which leads to a cascade of signals ultimately causing a rise in blood pressure. Neuromodulation of the renal nerves 120 (or renal denervation) removes or diminishes this response and facilitates a return to normal blood pressure.

The renal artery 80 has smooth muscle cells 130 that surround the arterial circumference and spiral around the angular axis θ of the artery. The smooth muscle cells 130 of the renal artery 80 have a longer dimension extending transverse (i.e., non-parallel) to the longitudinal axis LA of the renal artery 80. The misalignment of the lengthwise dimensions of the renal nerves 120 and the smooth muscle cells 130 is defined as “cellular misalignment.” This cellular misalignment of the renal nerves 120 and the smooth muscle cells 130 may be exploited to selectively affect renal nerve cells with a reduced effect on smooth muscle cells.

In FIG. 4a, the first portion 141 of the renal artery 80 includes a lumen 140 that extends lengthwise through the renal artery along the longitudinal axis LA. The lumen 140 is a generally cylindrical passage that allows the flow of oxygenated blood from the abdominal aorta to the kidney. The lumen 140 includes a luminal wall 150 that forms the blood-contacting surface of the renal artery 80. The distance D1 corresponds to the luminal diameter of lumen 140 and defines the diameter or perimeter of the blood flow lumen. A distance D2, corresponding to the wall thickness, exists between the luminal wall 150 and the renal nerves 120. The relatively healthy renal artery 80 may have an almost uniform distance D2 or wall thickness with respect to the lumen 140. The relatively healthy renal artery 80 may decrease substantially regularly in cross-sectional area and volume per unit length, from a proximal portion near the aorta to a distal portion near the kidney.

FIG. 4b illustrates the third portion 143 of the renal artery 80 including a lumen 140′ that extends lengthwise through the renal artery along the longitudinal axis LA. The lumen 140′ includes a luminal wall 150′ which forms the blood-contacting surface of the renal artery 80′. In some patients, the smooth muscle wall of the renal artery is thicker than in other patients, and consequently, as illustrated in FIG. 3b, the lumen of the third portion 143 of the renal artery 80 possesses a smaller diameter relative to the renal arteries of other patients. The lumen 140′, which is smaller in diameter and cross-sectional area than the lumen 140 pictured in FIG. 4a, is a generally cylindrical passage that allows the flow of oxygenated blood from the abdominal aorta to the kidney. A distance D2′ exists between the luminal wall 150′ and the renal nerves 120 that is greater than the distance D2 pictured in FIG. 4a.

FIG. 4c illustrates the diseased second portion 142 of the renal artery 80 including atherosclerotic changes. The second portion 142 includes a lumen 140″ that extends lengthwise through the renal artery along the longitudinal axis LA. Unlike the renal artery of a patient without atherosclerotic changes, as is pictured in FIGS. 4a and 4b, the lumen 140″ is an irregularly-shaped passage that may allow the flow of oxygenated blood from the abdominal aorta to the kidney at a reduced rate because the narrowed lumen creates a reduced cross-sectional area for blood flow. The lumen 140″ includes a luminal wall 150″ which forms the blood-contacting surface of the renal artery 80. The luminal wall 150″ is irregularly shaped by the presence of two atherosclerotic plaques 160, 170. A distance D3 exists between the luminal wall 150″ and the renal nerves 120 that is greater than the distance D2 pictured in FIG. 4a.

Earlier stages of atherosclerotic plaque formation are manifested as “fatty or lipid streaks” on luminal walls. These fatty streaks contain lipid-laden foam cells located in the subendothelial layer of the arterial intima. Additional intracellular and extracellular lipids accumulate at the site of the plaque during later plaque formation stages to cause raised lesions, such as the plaques 160, 170. In addition, smooth muscle and connective tissue cells may migrate into the plaque and proliferate within the plaque. Plaques damage the luminal surface of the artery, thereby weakening the artery and decreasing its elasticity. Luminal damage may also attract additional cells and extracellular materials to accumulate at or near the plaque. Over time, a plaque may calcify. As cells and extracellular materials accumulate, the luminal surface of the artery becomes irregular, as pictured in FIG. 4c, which may lead to the accumulation of blood platelets and thrombus formation. The American Heart Association has recognized several different stages of plaque formation starting from flat lipid streaks, through the visible raised lesions, and ending in a fully occluded artery. As such, atherosclerotic plaque formation is a continuum of events. As the plaques mature, the thickness of the arterial wall, and therefore the distance from the luminal wall to the nerves surrounding the artery, may expand.

In FIG. 4c, the atherosclerotic plaque 160 is a predominantly fatty plaque in the earlier stages of plaque formation. The atherosclerotic plaque 170 is a hardened, calcified plaque in the later stages of plaque formation. The distance D3 extending from the luminal wall 150″ to the renal nerves ranges in thickness along the circumferential and longitudinal span of the plaques 160, 170. Different types of plaques may possess different conductive and impedance properties, thereby affecting the amount, type, and duration of thermal energy that may be required to effectively modulate the nerves overlying the vessels in the region of the plaques.

FIG. 5 illustrates a thermal neuromodulation system 200 that is configured to deliver a thermal electric field to renal nerve fibers in order to achieve renal neuromodulation via heating and/or cooling according to one embodiment of the present disclosure. The system 200 includes a thermal basket catheter 210 comprising an elongate, flexible, tubular body 220 that is configured for intravascular placement and defines an internal lumen 225. The body 220 extends from a handle 230 along a longitudinal axis CA, which is coupled to an interface 240 by an electrical connection 245. The body 220 includes a proximal portion 250, and intermediate portion 255, and a distal portion 260. In FIG. 5, the thermal basket catheter 210 is pictured in an unexpanded condition. The proximal portion 250 may include shaft markers 262 to aid in positioning the catheter in the body of a patient. The intermediate portion 255 may include a guidewire exit port 265 from which a guidewire may emerge. The distal portion 260 may include several radiopaque markers 270, an imaging apparatus 280, and a distal tip 290. In addition, the distal portion 260 comprises an expandable structure 300 (not shown in FIG. 5) in an unexpanded condition within the body 220, located within the distal portion 260 and proximal to the distal tip 290. The imaging apparatus 280 is positioned on a proximal segment of the distal tip 290, which may be axially spaced from the rest of the body 220 along the longitudinal axis CA to reveal the expandable structure 300 in a gradually expanding condition.

The interface 240 is configured to connect the catheter 210 to a patient interface module or controller 310, which may include a guided user interface (GUI) 315. More specifically, in some instances the interface 240 is configured to communicatively connect at least the imaging apparatus 280 and the expandable structure 300 of the catheter 210 to a controller 310 suitable for carrying out intravascular imaging and thermal neuromodulation. The controller 310 is in communication with and performs specific user-directed control functions targeted to a specific device or component of the system 200, such as the thermal basket catheter 210, the imaging apparatus 280, and/or the expandable structure 300.

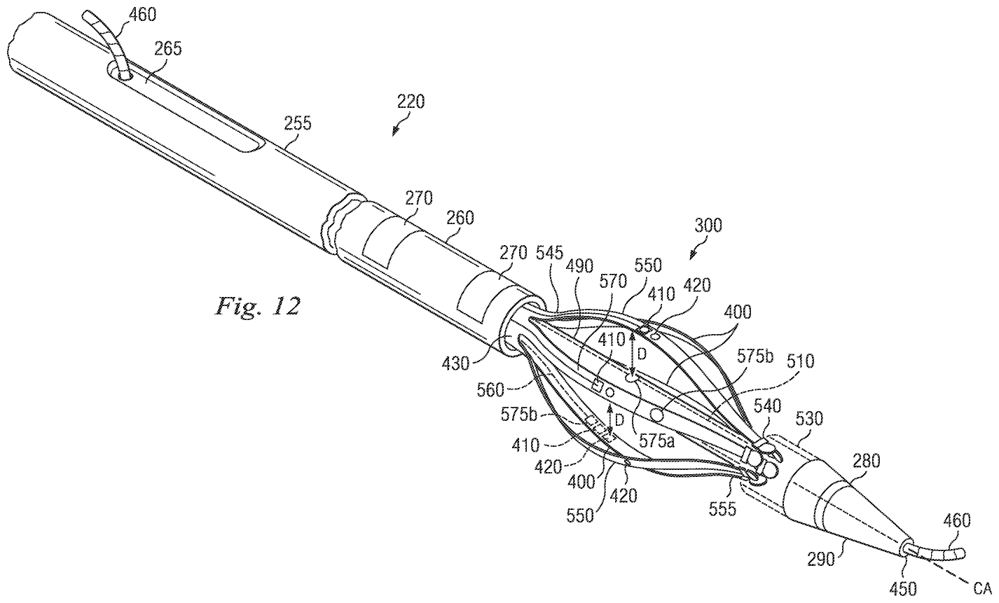

The interface 240 may also be configured to include a plurality of electrical connections, each electrically coupled to an electrode and/or a sensor on the expandable structure 300 via a dedicated conductor and/or a sensor cable (not shown), respectively, running through the body 220 as described in more detail below with respect to FIG. 12. Such a configuration allows for a specific group or subset of electrodes on the expandable structure 300 to be easily energized with either monopolar or bipolar energy, for example. Such a configuration may also allow the expandable structure 300 to transmit data from any of a variety of sensors via the controller 310 to data display modules such as the GUI 315 and/or the processor 320. The interface 240 may be coupled to the thermal electric field generator 325 via the controller 310, with the controller 310 allowing energy to be selectively directed to the portion of a luminal wall of the renal artery that is engaged by the expandable structure 300 while in an expanded condition.

The controller 310 may be connected to a processor 320, which is typically an integrated circuit with power, input, and output pins capable of performing logic functions, an imaging energy generator 322, and a thermal electric field generator 325. The processor 320 may include any one or more of a microprocessor, a controller, a digital signal processor (DSP), an application specific integrated circuit (ASIC), a field-programmable gate array (FPGA), or equivalent discrete or integrated logic circuitry. In some examples, processor 320 may include multiple components, such as any combination of one or more microprocessors, one or more controllers, one or more DSPs, one or more ASICs, or one or more FPGAs, as well as other discrete or integrated logic circuitry. The functions attributed to processor 320 herein may be embodied as software, firmware, hardware or any combination thereof.

The processor 320 may include one or more programmable processor units running programmable code instructions for implementing the thermal neuromodulation methods described herein, among other functions. The processor 320 may be integrated within a computer and/or other types of processor-based devices suitable for a variety of intravascular applications, including, by way of non-limiting example, thermal neuromodulation and intravascular imaging. The processor 320 can receive input data from the controller 310, from the imaging apparatus 280 and/or the expandable structure 300 directly via wireless mechanisms, or from the accessory devices 340. The processor 320 may use such input data to generate control signals to control or direct the operation of the catheter 210. In some embodiments, the user can program or direct the operation of the catheter 210 and/or the accessory devices 340 from the controller 310 and/or the GUI 315. In some embodiments, the processor 320 is in direct wireless communication with the imaging apparatus 280 and/or the expandable structure 300, and can receive data from and send commands to the imaging apparatus 280 and/or the expandable structure 300.

In various embodiments, processor 320 is a targeted device controller that may be connected to a power source 330, accessory devices 340, a memory 345, and/or the thermal electric field generator 325. In such a case, the processor 320 is in communication with and performs specific control functions targeted to a specific device or component of the system 200, such as the imaging apparatus 280 and/or the expandable structure 300, without utilizing user input from the controller 310. For example, the processor 320 may direct or program the imaging apparatus 280 and/or the expandable structure 300 to function for a period of time without specific user input to the controller 310. In some embodiments, the processor 320 is programmable so that it can function to simultaneously control and communicate with more than one component of the system 200, including accessory devices 330, a power source 340, and/or a thermal electric field generator 325. In other embodiments, the system includes more than one processor and each processor is a special purpose controller configured to control individual components of the system.

The power source 330 may be a rechargeable battery, such as a lithium ion or lithium polymer battery, although other types of batteries may be employed. In other embodiments, any other type of power cell is appropriate for power source 330. The power source 330 provides power to the system 200, and more particularly to the processor 320. The power source 330 may be an external supply of energy received through an electrical outlet. In some examples, sufficient power is provided through on-board batteries and/or wireless powering.

The various peripheral devices 340 may enable or improve input/output functionality of the processor 320. Such peripheral devices 340 include, but are not necessarily limited to, standard input devices (such as a mouse, joystick, keyboard, etc.), standard output devices (such as a printer, speakers, a projector, graphical display screens, etc.), a CD-ROM drive, a flash drive, a network connection, and electrical connections between the processor 320 and other components of the system 200. By way of non-limiting example, a processor may manipulate signals from the imaging apparatus 280 to generate an image on a display device, may coordinate aspiration, irrigation, and/or thermal neuromodulation, and may register the treatment with the image. Such peripheral devices 340 may also be used for downloading software containing processor instructions to enable general operation of the catheter 210, and for downloading software implemented programs to perform operations to control, for example, the operation of any auxiliary devices attached to the catheter 210. In some embodiments, the processor may include a plurality of processing units employed in a wide range of centralized or remotely distributed data processing schemes.

The memory 345 is typically a semiconductor memory such as, for example, read-only memory, a random access memory, a FRAM, or a NAND flash memory. The memory 345 interfaces with processor 320 such that the processor 320 can write to and read from the memory 345. For example, the processor 320 can be configured to read data from the imaging apparatus 280 and write that data to the memory 345. In this manner, a series of data readings can be stored in the memory 345. The processor 320 is also capable of performing other basic memory functions, such as erasing or overwriting the memory 345, detecting when the memory 345 is full, and other common functions associated with managing semiconductor memory.

The controller 310 may be configured to couple the imaging apparatus 280 to an imaging energy generator 322. In embodiments where the imaging apparatus 280 is an IVUS, the imaging energy generator comprises an ultrasound energy generator. Under the user-directed operation of the controller 310, the imaging energy generator 322 may generate a selected form and magnitude of energy (e.g., a particular energy frequency) best suited to a particular application. At least one supply wire (not shown) passing through the body 220 and the interface 240 connects the imaging apparatus 280 to the imaging energy generator 322. The user may use the controller 130 to initiate, terminate, and adjust various operational characteristics of the imaging energy generator 318.

The thermal electric field generator 325 may be configured to produce thermal energy, e.g. RF energy, that may be directed to the expandable structure 300 when it assumes an expanded condition. Under the control of the user or an automated control algorithm in the processor 320, the generator 325 generates a selected form and magnitude of thermal energy. The generator 325 may be utilized with any of the thermal basket catheters described herein for delivery of a thermal electric field with the desired field parameters, i.e., parameters sufficient to thermally induce renal neuromodulation via heating, cooling, and/or other mechanisms such as electroporation. It should be understood that the thermal basket catheters described herein may be electrically connected to the generator 325 even through the generator 325 is not explicitly shown or described with respect to each embodiment. The user may direct whether the expandable structure 300 is energized with monopolar or bipolar RF energy by using the controller 310 or programming the processor 320.

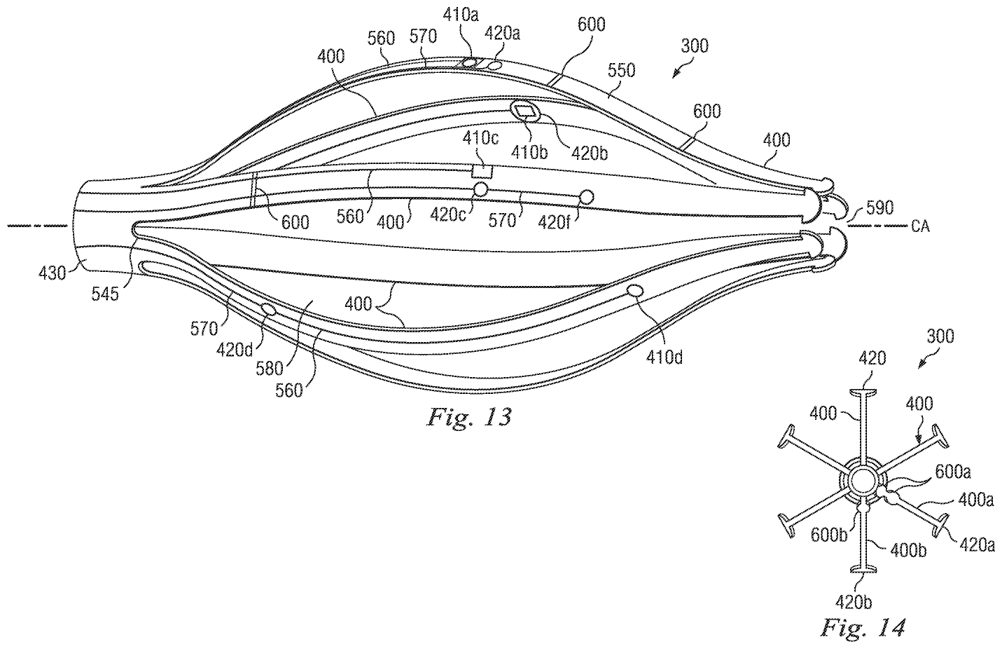

In the pictured embodiment, the generator 325 is located external to the patient. In other embodiments, the generator 325 may be positioned internal to the patient. In alternative embodiments, the generator may additionally comprise or may be substituted with an alternative thermal energy generator, such as, by way of non-limiting example, a thermoelectric generator for heating and/or cooling (e.g., a Peltier device) or a thermal fluid injection system for heating and/or cooling. For embodiments that provide for the delivery of a monopolar electric field via an electrode on the expandable structure 300, a neutral or dispersive ground pad or electrode 350 can be electrically connected to the generator 325. The control and direction of the energy supplied by the generator 325 will be described in further detail with respect to FIGS. 13 and 15.

FIG. 5 illustrates the thermal basket catheter 210 in an unexpanded condition according to one embodiment of the present disclosure. The thermal basket catheter includes the expandable structure 300 in an unexpanded condition positioned within the distal portion 260. As described above, the body 220 is an elongate flexible tube that defines the lumen 225 and the longitudinal axis of the catheter CA. The body 220 is configured to flex in a substantial fashion to traverse tortuous intravascular pathways and gain entrance to the renal arteries. The lumen 225 may be used for the delivery of thermal energy, for sensing various characteristics, and for imaging the vascular and neural anatomy. The lumen 225 may also be used as an access lumen for a guidewire. In some embodiments, the lumen 225 may be used for irrigation of a vessel lumen and aspiration of cellular debris, such as plaque material. In some embodiments, the body 220 includes more than one lumen. The lumen 225 will be described in further detail below with respect to FIGS. 8-10.

As described above, the proximal portion 250 may include shaft markers 262 disposed along the body of the catheter 210 that aid in positioning the catheter in the body of a patient. The shaft markers 262 may be positioned a specific distance from each other and comprise a measurement scale reflecting the distance of the marker 262 from the expandable structure 300. The proximal portion 250 may include any number of shaft markers 262 positioned a fixed distance away from the expandable structure 300 associated with a range of expected distances from the patient's skin surface at the point of catheter entry to the desired zone of thermal neuromodulation. For example, the shaft markers may be positioned, by way of non-limiting example, 1 millimeter from each other, 1 centimeter from each other, and/or 1 inch from each other. After initially positioned the expandable structure within the target vessel for neuromodulation, the user may utilize the shaft markers 262 to knowledgeably shift or reposition the catheter 210 along the intravascular target vessel to apply thermal energy at desired intervals along the target vessel before, after, or without employing imaging guidance. By noting the measurement and/or change in measured distance indicated by the shaft markers located immediately external to the patient's body as the catheter 210 is shifted, the user may determine the approximate distance and axial direction the expandable structure 300 has shifted within the patient's vasculature. In addition, the user may use the measurement and/or change in measured distance indicated by the shaft markers located immediately external to the patient's body to cross reference the intravascular position of the expandable structure 300 indicated by intravascular imaging. In some embodiments, the shaft markers 262 may be radiopaque or otherwise visible to imaging guidance. Other embodiments may lack shaft markers.

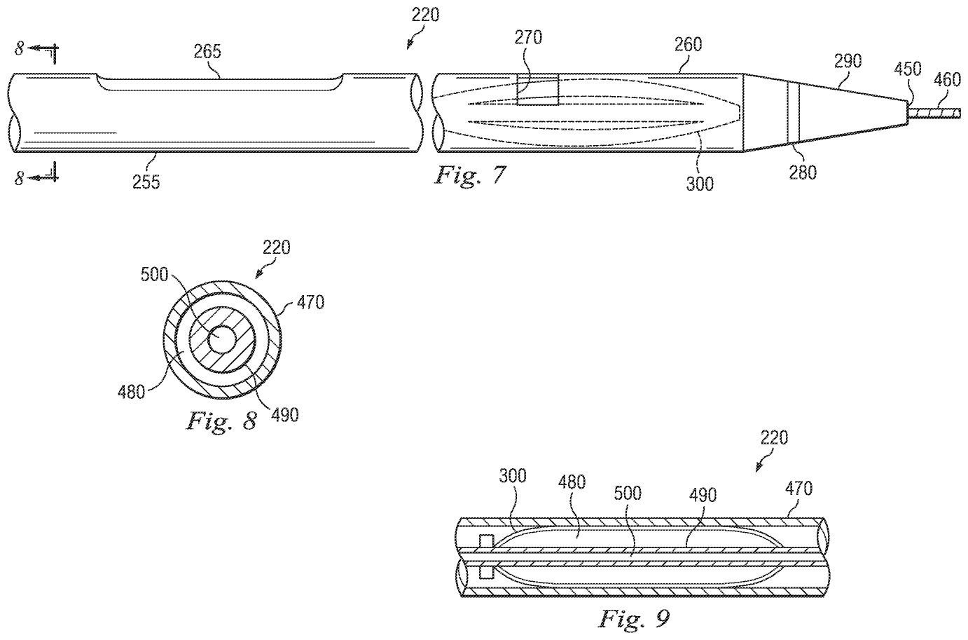

As described above, the intermediate portion 255 may include a guidewire exit port 265 from which a guidewire may emerge. The structure and function of the guidewire exit port 265 will be described in further detail below with respect to FIGS. 7-11.

The radiopaque markers 270 are spaced along the distal portion 260 at specific intervals from each other and at a specific distance from the distal tip 290. The radiopaque markers 270 may aid the user in visualizing the path and ultimate positioning of the catheter 210 within the vasculature of the patient. In addition, the radiopaque markers 270 may provide a fixed reference point for co-registration of various imaging modalities and treatments, including by way of non-limiting example, external imaging including angiography and fluoroscopy, imaging by the imaging apparatus 280, and thermal neuromodulation by the expandable structure 300. Other embodiments may lack radiopaque markers.

In the pictured embodiment, the imaging apparatus 280 is an intravascular ultrasound (IVUS) apparatus. More specifically, the imaging apparatus 280 pictured in FIG. 5 represents an ultrasound transducer. The entire IVUS apparatus may extend through the body 220 and include all the components associated with an IVUS module, such as a transducer(s), multiplexer(s), electrical connection(s), etc., for performing IVUS imaging. The imaging apparatus 280 of the pictured embodiment may utilize any IVUS configuration that allows at least a portion of the body 220 to be introduced over a guidewire. For example, in some instances, the imaging apparatus 280 utilizes an array of transducers (e.g., 32, 64, 128, or other number transducers) disposed circumferentially about the central lumen 225 of the body 220 in a fixed orientation. In other embodiments, the IVUS portion 118 is a rotational IVUS system. In some instances, the imaging apparatus 280 includes components similar or identical to those found in IVUS products from Volcano Corporation, such as the Eagle Eye® Gold Catheter, the Visions® PV8.2F Catheter, the Visions® PV 018 Catheter, and/or the Revolution® 45 MHz Catheter, and/or IVUS products available from other manufacturers. Further, in some instances the catheter 210 includes components or features similar or identical to those disclosed in U.S. Pat. Nos. 4,917,097, 5,368,037, 5,453,575, 5,603,327, 5,779,644, 5,857,974, 5,876,344, 5,921,931, 5,938,615, 6,049,958, 6,080,109, 6,123,673, 6,165,128, 6,283,920, 6,309,339; 6,033,357, 6,457,365, 6,712,767, 6,725,081, 6,767,327, 6,776,763, 6,779,257, 6,780,157, 6,899,682, 6,962,567, 6,976,965, 7,097,620, 7,226,417, 7,641,480, 7,676,910, 7,711,413, and 7,736,317, each of which is hereby incorporated by reference in its entirety.

In alternate embodiments, the imaging apparatus 280 may be or include, by way of non-limiting example, any of grey-scale IVUS, forward-looking IVUS, rotational IVUS, phased array IVUS, solid state IVUS, optical coherence tomography, or virtual histology. It is understood that, in some instances, wires associated with the imaging apparatus 280 extend along the length of the elongated tubular body 220 through the handle 230 and along electrical connection 245 to the interface 240 such that signals from the imaging apparatus 280 can be communicated to the controller 310. In some instances, the imaging apparatus 280 communicates wirelessly with the controller 310 and/or the processor 320.

In alternate embodiments, the imaging apparatus 280 may work in cooperation with or be substituted by an independent imaging catheter that is threaded through the lumen 225 of the catheter 210. In such embodiments, the independent imaging catheter may be axially moveable and rotational within the body 220 such that the imaging components of the imaging catheter may be positioned in a multitude of places along the longitudinal axis CA relative to the expandable structure 300. For example, a distal tip of the imaging catheter may be positioned proximal, within, or distal to the expandable structure 300 to gather image data about the surrounding tissue. In an embodiment where the imaging catheter is positioned within the expandable structure, the expandable structure may be constructed of translucent material or material that does not interfere with the data collection of the imaging catheter.

With reference to FIG. 5, in alternate embodiments, the imaging apparatus 280 may work in cooperation with or be substituted by a central imaging apparatus 355, which may be positioned on an exterior surface of an inner body 490 of the body 220. The central imaging apparatus 355 may be configured to function in substantially the same manner as the imaging apparatus 280.

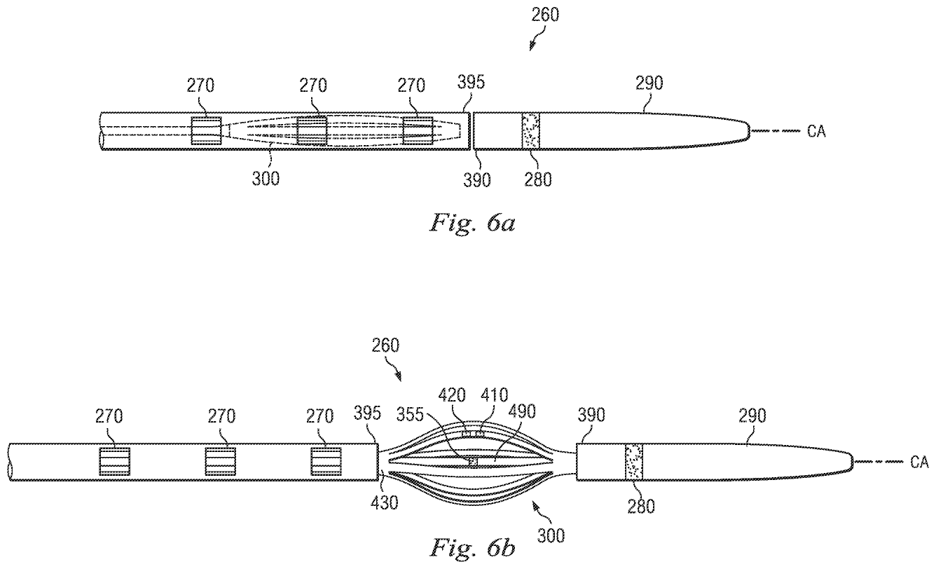

The proximal portion 250 of the body 220 connects to the handle 230, which is sized and configured to be securely held and manipulated by a user outside a patient's body. By manipulating the handle 230 outside the patient's body, the user may advance the body 220 of the catheter 210 through an intravascular path (as illustrated, for example, in FIG. 2) and remotely manipulate or actuate the distal portion 260. In the pictured embodiment, the handle 230 includes an elongated, slidable body actuator 360 positioned within an actuator recess 370. The body actuator 360 may be configured as any of a variety of elements, including by way of non-limiting example, a knob, a pin, or a lever, capable of manipulating or actuating the distal portion 260 to reveal the expandable structure 300. The operation of the body actuator 360 will be further described below with respect to FIGS. 6b and 7.

In alternate embodiments, the handle 230 may include a proximal port configured to receive fluid therethrough, thereby permitting the user to irrigate or flush the lumen 225 and/or the expandable structure 300. For example, the proximal port may include a Luer-type connector capable of sealably engaging an irrigation device such as a syringe. Image guidance using the imaging apparatus 280 or external imaging, e.g., radiographic, CT, or another suitable guidance modality, or combinations thereof, can be used to aid the user's manipulation of the catheter 210. In the pictured embodiment, the body 220 is integrally coupled to the handle 230. In other embodiments, the body 220 may be detachably coupled to the handle 230, thereby permitting the body 220 to be replaceable.

The catheter 210, or the various components thereof, may be manufactured from a variety of materials, including, by way of non-limiting example, plastics, polytetrafluoroethylene (PTFE), polyether block amide (PEBAX), thermoplastic, polyimide, silicone, elastomer, metals, such as stainless steel, titanium, shape-memory alloys such as Nitinol, and/or other biologically compatible materials. In addition, the catheter 210 may be manufactured in a variety of lengths, diameters, dimensions, and shapes. For example, in some embodiments the elongated body 220 may be manufactured to have length ranging from approximately 115 cm-155 cm. In one particular embodiment, the elongated body 220 may be manufactured to have length of approximately 135 cm. In some embodiments, the elongated body 220 may be manufactured to have a transverse dimension ranging from about 1 mm-2.67 mm (3 Fr-8 Fr). In one embodiment, the elongated body 200 may be manufactured to have a transverse dimension of 2 mm (6 Fr), thereby permitting the catheter 210 to be configured for insertion into the renal vasculature of a patient. These examples are provided for illustrative purposes only, and are not intended to be limiting.

FIG. 6a illustrates at least a segment of the distal portion 260 of the thermal basket catheter 210 in an unexpanded condition according to one embodiment of the present disclosure. In some instances, the thermal basket catheter 210 includes components or features similar or identical to those disclosed in U.S. Patent Application Publication No. US2004/0176699, which is hereby incorporated by reference in its entirety. In the pictured embodiment, the distal tip 290 is positioned against the remainder of the body along the longitudinal axis CA, and the expandable structure 300 is compressed within the lumen in an unexpanded condition. The distal portion 260 includes a distal connection part 390, which is the proximal-most part of the distal tip 290, and a proximal connection part 395, which abuts the distal connection part 390 when the catheter 210 is in an unexpanded condition. In the pictured embodiment, the imaging apparatus 280 is positioned distal to the distal connection part 390. Additionally or alternatively, the imaging apparatus may be positioned proximal to the proximal connection part 395.

FIG. 6b illustrates at least a segment of the distal portion 260 of the thermal basket catheter 210 in an unexpanded condition according to one embodiment of the present disclosure. In the pictured embodiment, the distal tip 290 is moved distally away from the remainder of the body along the longitudinal axis CA to allow the expandable structure 300 to emerge from the lumen and assume an expanded condition. Specifically, the distal connection part 390 is separated axially away from the proximal connection part 395 along the axis CA. As further described below with respect to FIGS. 7-11, the user may transition the catheter 210 from an unexpanded condition to an expanded condition by manipulating the body actuator 360 within the actuator recess 370 to cause the distal tip 290 to move distally away from the remainder of the body 220. In the pictured embodiment, the expandable structure 300 is shown in a deployed and expanded condition wherein at least one support arm 400 has expanded outwardly. The expandable structure 300 includes six flexible support arms 400. In other embodiments, the expandable structure may include any number of support arms 400. At least one electrode 410 and at least one sensor 420 may be positioned on at least one of the support arms 400. The at least one electrode 410 and at least one sensor 420 will be described in further detail below with reference to FIGS. 12 and 13.

The support arms 400 may be manufactured from a variety of biocompatible materials, including, by way of non-limiting example, superelastic or shape memory alloys such as Nitinol, and other metals such as titanium, Elgiloy®, and/or stainless steel. The support arms 400 could also be made of, by way of non-limiting example, polymers or polymer composites that include thermoplastics, resins, carbon fiber, and like materials. In the illustrated embodiment, the support arms 400 are secured to a deployment support member 430, which may be secured to an interior component of the body 220 (as shown in FIGS. 8 and 9) in a variety of ways, including by way of non-limiting example, adhesively bonded, laser welded, mechanically coupled, or integrally formed. In alternate embodiments, the support arms 400 may be secured to an interior component of the body 220 directly, thereby eliminating the need for a deployment support member 430.

FIG. 7 illustrates the thermal basket catheter 210 in an unexpanded condition prior to deployment of the expandable structure 300 according to one embodiment of the present disclosure. More specifically, FIG. 7 illustrates a segment of the body 220 in an unexpanded condition, including a segment of the intermediate portion 255 and a segment of the distal portion 260. The expandable structure 300 is positioned proximate to the distal portion 260 of the catheter 210. As mentioned above, the intermediate portion 255 of the body may include the guidewire exit slot 265 thereon. The distal tip 290 of the distal portion 260 may include at least one guidewire port 450 capable of receiving a guidewire 460 therein.

FIG. 8 illustrates a transverse cross-sectional view of the body 220 of the thermal basket catheter 210 as taken along the lines 8-8 of FIG. 7 according to one embodiment of the present disclosure. FIG. 9 illustrates the expandable structure 300 in a non-deployed and unexpanded condition according to one embodiment of the present disclosure. As shown in FIGS. 8 and 9, the elongated body 220 may include an outer sleeve 470 forming a sleeve lumen 510 and housing an inner body 490 therein. In one embodiment, the outer sleeve 470 may be manufactured from a material, such as PEBAX, having a wall thickness of about 0.0127 mm to about 0.0762 mm. In another embodiment, the outer sleeve 470 has a wall thickness of about 0.0381 mm to about 0.0635 mm. These ranges are provided for illustrative purposes only, and are not intended to be limiting.

As shown in FIG. 9, the expandable structure 300 of the catheter 210 may be positioned within the sleeve lumen 5100 formed by the outer sleeve 470 prior to deployment. As shown, the expandable structure 300 may be compressed inwardly by an inner surface of the outer sleeve 470 and located within the sleeve lumen 510. In an alternate embodiment, the elongated body 220 may be manufactured without an outer sleeve. The inner body 490 defines an internal passage 500 therein. In the illustrated embodiment, an internal passage 500 is formed within the inner body 490, however, the internal passage may not be present in some embodiments. In another embodiment, the inner body 490 may define a plurality of internal passages therein. The internal passage 500 formed in the inner body 490 may be in communication with the guidewire port 450 located on the distal tip 290 and may be capable of receiving the guidewire 460 therein (as shown in FIG. 7).

As shown in FIG. 7, the expandable structure 300 is positioned proximate to the distal portion 260 of the catheter 210. Returning to FIG. 9, the expandable structure is compressed inwardly by an inner surface of the outer sleeve 470. The outer sleeve 470 may be in communication with or attached to the elongated body actuator 360 positioned within the actuator recess 370 located on the handle 230 (as illustrated in FIG. 5). The rearward movement of the elongated body actuator 360 within the actuator recess 370 results in the outer sleeve 470 retracting rearwardly from the distal tip 290, thereby permitting the expandable structure 300 to expand radially and assume an expanded condition.

In an alternate embodiment, the outer sleeve 470 may remain stationary while the inner body 490 may be capable of moving in telescopic relation thereto. For example, the inner body 490 may communicate with the elongated body actuator 360 positioned within the actuator recess 370 located on the handle 230 (as illustrated in FIG. 5). The forward movement of the elongated body actuator 360 within the actuator recess 370 results in the inner body 490 extending distally from the handle 230 (as illustrated in FIG. 6), thereby advancing the expandable structure 300 beyond the outer sleeve 470 and permitting the expandable structure 300 to expand radially (to contact the luminal wall of an artery, for example).

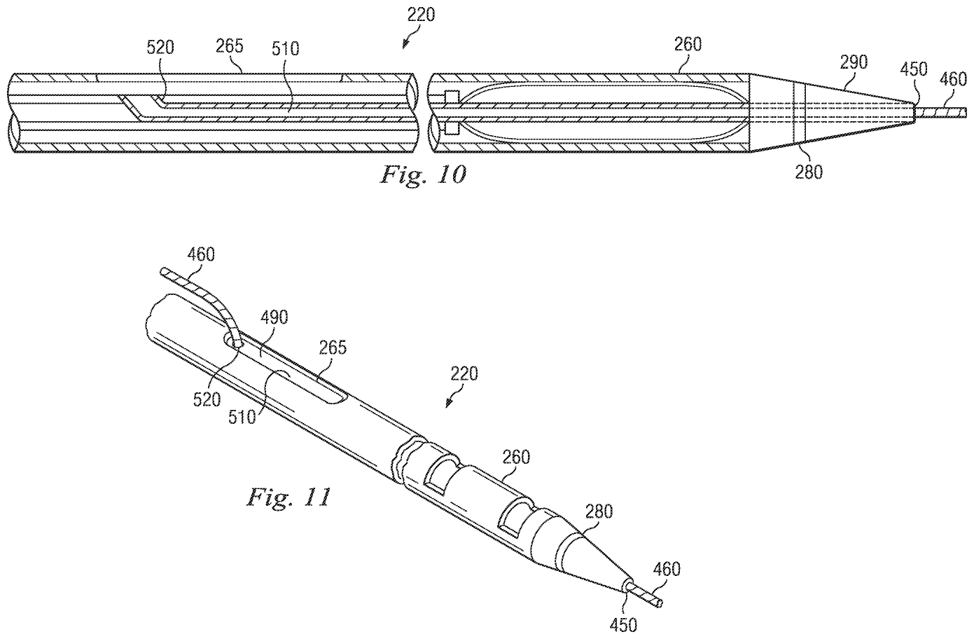

Both FIGS. 10 and 11 illustrate a distal segment of the thermal basket catheter 210 in an unexpanded condition according to one embodiment of the present disclosure. As shown in FIG. 10, a guidewire lumen 510 may be secured to the guidewire port 450 on the distal tip 290. A proximal end of the guidewire lumen 510 communicates with a guidewire exit port 520 in the inner body 490, thereby permitting the guidewire port 450 to communicate with the guidewire exit slot 265. The guidewire lumen 510 may be secured to the guidewire port 450 using, by way of non-limiting example, adhesives or bonding agents, mechanical couplers, pins, snap-fit devices, and other coupling devices known in the art.

As a shown in FIG. 11, the guidewire 460 may be introduced into the guidewire port 450 and made to traverse the guidewire lumen 510 within the inner body 490, exiting the thermography catheter 210 through the guidewire exit port 520 positioned in the guidewire exit slot 265. The guidewire exit slot 265 may be formed at a variety of distances along the elongated body 220. In some embodiments the distance between the guidewire port 450 and the guidewire exit slot 265 ranges from about about 10 cm to about 20 cm. For example, in one embodiment the distance between the guidewire port 450 and the guidewire exit slot 265 ranges from about 10 cm to about 12 cm. These examples are provided for illustrative purposes only, and are not intended to be limiting.

FIG. 12 illustrates the thermal basket catheter 210 in an expanded condition according to one embodiment of the present disclosure wherein the distal tip 290 has been moved axially away from the remainder of the distal portion 260 and at least one of the support arms 400 has expanded outwardly. The support arms 400 may be manufactured in any of a variety of shapes, including by way of non-limiting example, arcuate shapes, bell shapes, smooth shapes, and step-transition shapes. The support arms include a proximal section 545, a medial section 550, and a distal section 555. The proximal section 545 may be capable of coupling the expandable structure 300 to the body 220 or the inner body 490. The medial section 550 is configured to be positioned proximate to or in contact with a vessel luminal wall. The distal section 555 couples each arm 400 to a support arm retainer 540 positioned on an exterior of the inner body 490.

The transverse or cross-sectional profile of the support arms 400 may be manufactured in any of a variety of shapes, including oblong, ovoid, and round. In some embodiments, the cross-sectional profile of the support arm includes rounded or atraumatic edges to minimize damage to an artery or a tubular structure through which the expandable structure 300 may travel.

In one embodiment, the proximal sections 545 of the support arms 400 may be coupled to the deployment support member 430 using an adhesive, such as, by way of non-limiting example, Loctite 3311 adhesive or any other biologically compatible adhesive. In an alternate embodiment, the expandable structure 300 may be manufactured by laser cutting or forming the at least one support arm 50 from a substrate. For example, any number of support arms 400 may be laser cut within a Nitinol tube or cylinder, thereby providing a slotted expandable body. The support arms 400 may be fabricated from a self-expanding material biased such that the medial section 550 expands into contact with the vessel luminal wall upon expanding the catheter 210. In some embodiments, the one or more support arms 400 may be formed in a deployed state as shown in FIG. 12 wherein at least one support arm 400 is flared outwardly from the longitudinal axis CA of the catheter 210.

In the illustrated embodiment, the guidewire lumen 510, capable of receiving the guidewire 460 therein, longitudinally traverses the expandable structure 300. The guidewire lumen 510 is in communication with the guidewire port 450 on the distal portion 260 and guidewire exit slot 265 located on the elongated body 220. In an alternate embodiment, the guidewire lumen 510 may be in communication with the guidewire port 450 on the distal tip 290 and/or a proximal port located on the handle 230 (shown in FIGS. 4 and 5). In the illustrated embodiment, a retainer sleeve 530 is positioned over a distal section of the support arms 400 to provide a transition between the distal tip 290 and the support arms 400. As shown, the retainer sleeve 530 is positioned over the support arm retainers 540, thereby preventing the support arm retainers 540 from contacting the vessel wall 90 (see FIG. 12) and causing trauma to the vessel luminal wall (not shown), damaging the support arm retainers 540, or both. Other embodiments may lack a retainer sleeve.

During manufacture, the at least one support arm 400 is formed to assume a deployed position in a relaxed state as shown in FIG. 12, wherein the medial section 550 of the support arm 400 is flared outwardly a distance D from the longitudinal axis CA of the catheter 210. The application of force to the apex of the medial section 550 of the support arm 400 decreases the curvature of the support arm 400 resulting in a corresponding decrease in the distance D.

The at least one electrode 410 may be positioned on the medial section 550 of at least one of the support arms 400, thereby enabling the electrode 410 the sensor 420 to contact or approximate the vessel luminal wall. At least one electrode cable 560 connects each electrode 410 to the interface 240 and/or the thermal electric field generator 325. The at least one electrode 410 will be described in further detail below in reference to FIG. 13.

The at least one sensor 420 may be positioned on the medial section 550 of at least one of the support arms 400, thereby enabling the sensor to contact or approximate the vessel luminal wall. At least one sensor cable connects each sensor 420 to the sensor coupler 380 and/or the interface 240. The at least one sensor 420 will be described in further detail below in reference to FIG. 13.

The expandable structure 300 may include at least one ancillary sensor 575 thereon. As shown in FIG. 12, the ancillary sensor 575a may be positioned on an exterior surface of the inner body 490. In the alternative, at least one ancillary sensor 575b may be positioned on at least one support arm 400. Exemplary ancillary sensors 575 include, without limitation, ultrasonic sensors, flow sensors, thermal sensors, blood temperature sensors, electrical contact sensors, conductivity sensors, electromagnetic detectors, pressure sensors, chemical or hormonal sensors, pH sensors, and infrared sensors. For example, in one embodiment the ancillary sensor 575a may comprise a blood sensor positioned on the guidewire lumen 510 in the bloodstream as shown in FIG. 12, thereby permitting the sensors 420 located on the support arms 400 to measure the vessel wall temperature while simultaneously the ancillary sensor 575a measures blood temperature within the vessel. In another embodiment, the ancillary sensor 575b may comprise a pressure sensor positioned on the support arm 400 proximate to the electrode 410 and/or encircling the electrode 410. The ancillary pressure sensor 575b may detect the pressure with which the proximate electrode 410 is contacting the vessel wall, thereby allowing the user to determine whether the electrode 410 is effectively contacting the vessel wall to ensure adequate energy transfer and neuromodulation.

In the embodiment illustrated in FIG. 12, each support arm 400 is coupled by its distal section 555 to inner body 490 using the support arm retainer 540, thereby permitting each support arm 400 to move independently relative to the inner body 490 and the other support arms 400. The ability of the support arms 400 to independently move within the support arm retainer 540 results in the creation of an expandable structure 300 offering flexibility, while permitting the support arms 400 to remain in contact with a vessel wall (not shown) when traversing a tortuous or curved pathway, such as may be found in the renal arteries. More particularly, when the expandable structure 300 is in a non-deployed state, the ability of the support arms 400 to move independently of each other in an axial direction reduces shear resistance and results in a more flexible catheter than a catheter wherein the axial movement is coupled or otherwise restricted. In addition, when the expandable structure 300 is in a deployed and expanded state, the ability of the support arms 400 to move independently facilitates contact of each of the support arms 400 with the vessel wall without applying excessive force thereto, thereby decreasing or eliminating the likelihood of injury to the vessel. Maximizing contact of each of the support arms 400 with the vessel wall in turn maximizes contact of sensors 420 with the vessel wall, which can be important in some embodiments for obtaining accurate sensor readings.

Referring again to FIG. 12, the ability of support arms 400 to move independently with respect to the inner body 490 and the other support arms 400 results in the formation of a flexible expandable structure 300 capable of traversing tortuous vessel pathways. The support arms 400 of the expandable structure 300 may be manufactured in a variety of shapes, lengths, widths, and thickness to promote the flexibility of the individual support arms 400. A high degree of flexibility of the support arms helps to ensure the atraumatic deployment and movement of the expandable structure 300 within a vessel lumen or tubular structure. For example, in one embodiment the support arms 400 may have a length of about 5 mm to about 26 mm, and more specifically, a length of about 10 mm to about 16 mm. Similarly, the support arms 400 may be manufactured from a material having a thickness of about 0.0381 mm to about 0.1778 mm. More specifically, in one embodiment, the support arms 400 have a thickness of about 0.0635 mm to about 0.1143 mm. These ranges are provided for illustrative purposes only, and are not intended to be limiting.

FIG. 13 illustrates the expandable structure 300 removed from the catheter 210 and in an expanded condition according to one embodiment of the present disclosure. The expandable structure 300 may be generally hollow in design and may define an expandable body passage 590 capable of receiving the guidewire 32 or the inner body 490 therethrough (see FIG. 12). In some embodiments, the expandable structure 300 may be sized and configured for expansion, manipulation, and use within a renal artery. The expandable structure 300 may include any number of support arms 400 separated by one or more spaces 580. The arms 400 may be structurally supported with an insulated material such as, by way of non-limiting example, an ultraviolet cure or heat shrink sleeve, polyethelene, Nylon™, or the like. In the illustrated embodiment, the support arms 400 are symmetrically positioned around the expandable body passage 590. In an alternate embodiment, the support arms 400 are asymmetrically positioned around the expandable body passage 590. As stated above, the expandable structure 300 may be manufactured from a variety of materials, including, for example, shape memory alloys such as Nitinol, metals such as stainless steel and titanium, polymers, composite materials, and like materials. In one embodiment, the expandable structure 300 may be formed from a Nitinol hypodermic tube having at least one space 580 formed therein, thereby defining at least one support arm 400 thereon.

Each of the support arms 400 includes at least one electrode 410 and at least one corresponding electrode cable 560 thereon. The electrodes 410 may comprise individual electrodes (i.e., independent contacts), a segmented electrode with commonly connected contacts, or a single continuous electrode. The electrode cable 560 extends proximally from the electrode 410. The electrode 410 may comprise a raised component or a flat component on the support arm 400. The electrode 410 and/or the electrode cable 560 may be coupled to the support arm 400 using any of a variety of known connection methods, including by way of non-limiting example, welding, adhesive, and/or mechanical fasteners. For example, in one embodiment, the electrode 410 may be adhesively bonded to the support arm 400 using Loctite 3311 or any other biologically compatible adhesive. In some embodiments, the electrode 410 may be integrally formed with the support arm 400. Furthermore, all of a portion of the electrode may be coated or plated with gold, or a material having like properties, such as, by way of non-limiting example, silver or an alloy of copper, to improve radiopacity and/or conductivity without adversely diminishing the flexibility of the expandable structure 300.

At least one electrode 410 is positioned on the medial section 550 of the support arm 400, thereby permitting the electrode 410 to be positioned proximate to or in contact with a vessel luminal wall when the expandable structure is deployed and in an expanded condition. Any remaining electrodes 410 may be located at any position along the length of the support arm 400. The expandable structure 300 may include support arms 400 including any variation or pattern of electrode distribution among the individual support arms. Depending upon the desired application of the thermal basket catheter 210, the expandable structure 300 may have an identically configured pattern of electrodes 410 on the support arms 400, or a varying pattern of electrodes 410 on the support arms 400. For example, in the pictured embodiment, the electrodes 410a, 410b, and 410c are positioned on the medial section 550, while the electrode 410d is positioned on the distal section 555 of the support arm 400.

Each electrode 410 is electrically coupled to the field generator 325, which is disposed external to the patient, for the delivery of a thermal electric field for the heating of target neural fibers. In the pictured embodiment, each electrode 410 is connected to the corresponding electrode cable 560, which traverses the length of the support arm 400 from the electrode 410 to the interface 240 and/or the thermal electric field generator 325. In some embodiments, the electrode cable 560 may be selectively insulated such that only a selective portion of the electrode cable, e.g., a distal tip of the cable, may be electrically active. In alternate embodiments, several electrodes may be coupled to the field generator using one or more shared electrode cables. In other embodiments, the electrodes may communicate with the field generator 325 via wireless means.

Each of the support arms 400 includes at least one sensor 420 and at least one corresponding sensor cable 570 thereon. The sensor 420 may comprise a raised component or a flat component on the support arm 400. The sensor cable 570 extends proximally from the sensor 420. The sensor 420 and/or the sensor cable 570 may be coupled to the support arm 400 using any of a variety of known connection methods, including by way of non-limiting example, welding, adhesive, and/or mechanical fasteners. For example, in one embodiment, the sensor 420 may be adhesively bonded to the support arm 400 using Loctite 3311 or any other biologically compatible adhesive. In some embodiments, the sensor 420 may be integrally formed with the support arm 400. For example, in some embodiments, at least one sensor 420 may be comprised of flexible circuits integrated into at least one support arm 400. The flexible circuit may be comprised of polymer thick film flex circuit that incorporates a specially formulated conductive or resistive ink that is screen printed onto the flexible substrate to create the thermal sensor circuit patterns. This substrate is then adhered to the surface of each of the support arms 400. In an alternate embodiment, the substrate can be adhered to independently expandable, resilient body arms which are not part of an expandable structure 300. The independent sensor body can be provided with the appropriate number of body arms, such as four, five, six, or more.

At least one sensor cable connects each sensor 420 to the sensor coupler 380 and/or the interface 240. In alternate embodiments, several sensors may be coupled to the sensor coupler 380 and/or the interface 240 using one or more shared sensor cables, as illustrated by sensors 420c and 420f. In other embodiments, the sensors 420 may communicate with the sensor coupler 380, interface 240, and/or processor 320 via wireless means. The at least one sensor cable 570 may traverse the elongated body 220 through the sleeve lumen 480, the internal passage 500 (as illustrated in FIG. 10), or both. In some embodiments, a single cable may convey thermal energy to the electrode 410 and convey data from the sensor 420.

Exemplary sensors 420 include, without limitation, ultrasonic sensors, flow sensors, thermal sensors, such as thermocouples, thermistors and infrared sensors, pressure sensors, electrical contact sensors, conductivity and/or impedance sensors, electromagnetic detectors, fluid flow sensors, electrical current sensors, tension sensors, chemical or hormonal sensors (capable of detecting the concentration or presence/absence of various gases, ions, enzymes, proteins, metabolic products, etc.), and pH sensors. For example, the sensor 420 may comprise a thermocouple or other type of temperature sensor for monitoring the temperature of the target tissue, the non-target tissue, the surrounding blood, the electrodes 410, or any other part of the expandable structure 300. In one embodiment, the thermocouple may be capable of detecting thermal discontinuities or variations in vessel wall temperature, thereby providing a thermal basket catheter capable of locating inflamed or vulnerable plaques on the luminal wall of a blood vessel in vivo. The expandable structure 300 may contain any of a variety of sensor types within a single embodiment. As a result, the catheter 210 may be capable of simultaneously examining a number of different characteristics of the target tissue, the surrounding environment, and/or the catheter 210 itself within the body of a patient, including, for example, vessel wall temperature, blood temperature, electrode temperature, fluorescence, luminescence, flow rate, and flow pressure.

The at least one sensor 420 may be located at any position along the length of the support arm 400. In some embodiments, the at least one sensor 420 may be located proximate to the electrode 410 on the support arm 400, as illustrated by sensors 420a and 420c. In the same or alternate embodiments, at least one sensor 420 may be positioned within or surrounding the electrode 410, as illustrated by sensor 420b. As shown in FIG. 13 by sensors 420a and 420c, the sensor 420 may be positioned on or near the apex of the curved support arms 400 when the expandable structure 300 is deployed in an expanded state, thereby permitting the sensors to contact a vessel luminal wall. In some embodiments, the sensor 420a, b, and/or c may comprise a pressure sensor(s) that may detect the pressure with which the proximate electrode 410 is contacting the vessel wall, thereby allowing the user to determine whether the electrode 410 is effectively contacting the vessel wall to ensure adequate energy transfer and neuromodulation. In some embodiments, as illustrated by sensors 420d and 420f, the at least one sensor 420 may be positioned on the support arms 400 at any radial distance less than the radial distance of the apex of the curved support arms 400 relative the longitudinal axis CA when the expandable structure 300 is in a deployed state, thereby preventing the at least one sensor from contacting a vessel luminal wall when the expandable structure 300 is deployed to an expanded state.

Depending upon the desired application of the thermal basket catheter 210, the expandable structure 300 may have an identically configured pattern of electrodes 410 and sensors 420 on the support arms 400, or a varying pattern of electrodes 410 and sensors 420 on the support arms 400. For example, in the pictured embodiment, the sensors 420a, 420b, and 420c are positioned on the medial section 550, while the sensor 420d is positioned on the proximal section 545 of the support arm 400.

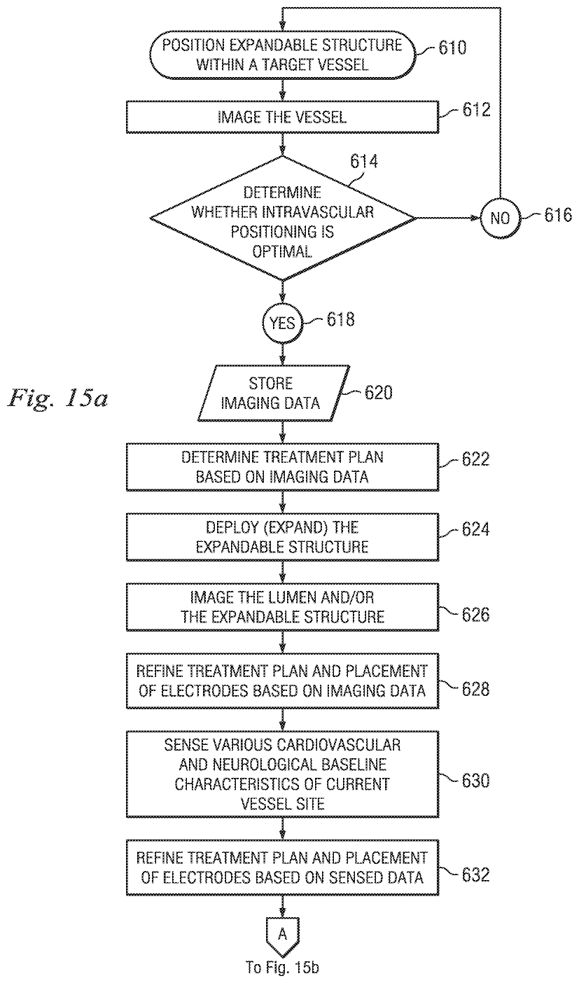

In some embodiments, radiopaque markers 600 may be positioned along the length of the support arms 400, aiding in the placement and visualization of the thermal basket catheter 210. In some embodiments, as shown in FIG. 14, individual support arms 400 may carry a distinctive pattern or shape of radiopaque markers 600 to enable the user to distinguish individual support arms in the image data gathered from the imaging apparatus 280 and/or external imaging. For example, the support arm 400a carries two distinctively shaped radiopaque markers 600a while the support arm 400b carries a distinctively shaped radiopaque marker 600b. In other embodiments, alternatively or additionally, the electrodes 410 and/or the sensors 420 are radiopaque or coupled to radiopaque markers (not shown).

The electrodes 410 may be configured to provide differential or selective heating of the vessel luminal wall, wherein individual electrodes may be selectively activated to convey thermal energy to the vessel luminal wall while other electrodes on the same or different support arm 400 are not activated and do not provide thermal energy. In addition, individual electrodes 410 may be configured to convey different amounts of thermal energy to different parts of the vessel luminal wall. Furthermore, the electrodes 410 may be configured to provide a bipolar signal, or the electrodes may be used together or individually in conjunction with the separate patient ground pad or electrode 350. As illustrated in FIG. 13, the electrodes 410 are distributed circumferentially about the axis CA in an array, with adjacent electrodes being slightly axially offset, preferably being staggered or alternating between more proximal and more distal positions on the medial section 550. This arrangement allows bipolar energy to be directed between adjacent circumferential electrodes, between adjacent “distal” electrodes, between adjacent “proximal” electrodes, and the like.

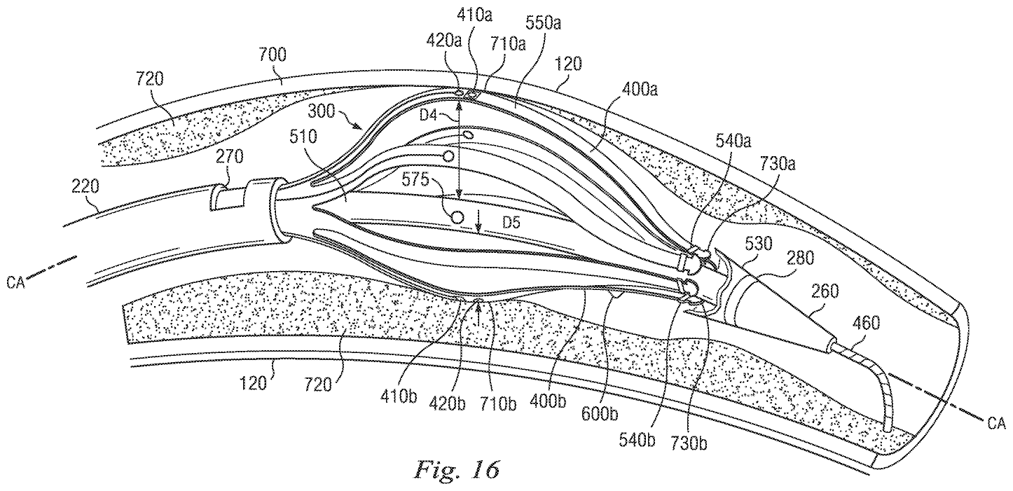

FIGS. 15a and 15b provide a schematic flowchart illustrating methods of delivering and controlling the thermal neuromodulation to renal vessels. With reference to FIGS. 2, 15a, and 16, step 610 comprises the user initiating a thermal neuromodulation procedure by positioning the thermal basket catheter 210 within the renal artery 80. Prior to insertion of the catheter 210, the guidewire 460 (as illustrated in FIG. 7) may be introduced into the arterial vasculature of a patient using standard percutaneous techniques. Once the guidewire 460 is positioned within the target blood vessel, which is the left renal artery 80 in the illustrated embodiment, the catheter 210 may be introduced into the arterial vasculature of a patient over the guidewire 460 and advanced to the area of interest. In the alternative, the catheter 210 may be coupled to the guidewire 460 external to the patient and both the guidewire 460 and the catheter 210 may be introduced into the patient and advanced to an area of interest simultaneously. The catheter 210 may include IVUS or other imaging apparatuses 280 (as shown in FIG. 16) thereon, thereby permitting the user to precisely position the catheter 210 within the blood vessel by using in vivo, real-time intravascular imaging. Additionally or alternatively, the user may utilize external imaging, such as, by way of non-limiting example, fluoroscopy, ultrasound, CT, or Mill, to aid in the guidance and positioning of the catheter 210 within the patient's vasculature. The external and intravascular images may be co-registered to each other for side-by-side or composite display of the images.

The catheter 210 is positioned within the renal anatomy such that the expandable structure 300, which is disposed in an unexpanded condition within the outer sleeve 470 (as shown in FIG. 9) when introduced the patient's vasculature, is positioned proximal to the target area of interest, including, by way of non-limiting example, renal artery 80, the inferior renal vessels 115, and/or the abdominal aorta 90. Prior to expanding the expandable structure 300, at step 612, the user may utilize the imaging apparatus 280 and/or the central imaging apparatus 355 to obtain intravascular images of the target area and area immediately surrounding the target area. The imaging apparatus 280 and/or the central imaging apparatus 355 may obtain images of the vessel wall concentrically about the catheter 210 so as to measure the thickness of the vessel wall in the target area of interest. In some cases, the imaging data may allow identification and/or characterization of the atherosclerotic changes, plaques, tissues, lesions, and the like from within the blood vessel. For example, the data may lead to a determination of the optimal intravascular location for the application of thermal neuromodulation.

At step 614 of FIG. 15a, the processor 320 and/or the user may analyze the intravascular images obtained by the imaging apparatus 280 and/or the central imaging apparatus 355 to determine whether the renal artery 80 possesses atherosclerotic changes or other disease processes of the vessel wall in the target area of interest. As illustrated in FIG. 4c, distance D3 exists between the luminal wall 150″ and the renal nerves 120 in the area of an atherosclerotic plaque that is greater than the distance D2 that exists between a healthy vessel wall 150 and the renal nerves 120 pictured in FIG. 4a. At step 616, if the user and/or the processor 320 determines that the vessel area immediately surrounding the expandable structure 300 is not the optimal site for thermal neuromodulation within the vessel based on the positional imaging data based on the imaged intraluminal vessel contours, wall thicknesses, and plaque types (as shown in FIG. 3), the user and/or the processor 320 may return to step 610 and reposition the catheter 210 into a portion of the artery 80 containing less plaque or having a thinner wall.

For example, if the intravascular imaging suggests the presence of eccentric atherosclerotic plaques or thickening along the length of the renal artery 80, as shown by portion 142 in FIGS. 3 and 4c, the processor 320 and/or the user may reposition the catheter 210 in an optimal area having the thinnest intra-to-extravascular distance across the vessel wall (as shown by portion 141 in FIGS. 3 and 4a). For example, the intravascular imaging may reveal the presence of calcified changes in the vessel wall, which can hinder the transfer of energy through the vessel wall to the target nerves. Ultimately, the user and/or the processor 320 may direct more thermal energy to the electrodes 410 positioned adjacent the thicker and/or more calcified portions of the plaque than those positioned against thinner portions of the plaque or the healthier portions of the vessel wall, thereby enabling the appropriate amount of thermal energy to reach the target renal nerves.

Once the user and/or the processor 320 have determined at step 618 that the catheter 210 is positioned in the optimal location for neuromodulation within the vessel, at step 620, the processor 320 and/or the user may record or store the intravascular position of the catheter 210 within the renal artery 80 or the abdominal aorta 90 relative to the renal ostia 92. At step 622, the user may use this positional data about the intraluminal characteristics of the optimal vessel site, including, by way of non-limiting example, the intra-extravascular or intra-extraluminal distance, the wall thickness, and/or the type of atherosclerotic plaque, to plan the current treatment procedure and/or repeat treatment procedures for the same intravascular site. Throughout the neuromodulation procedure, the user and/or the processor 320 may store imaged and/or sensed data.

At step 624 of FIG. 15a, after assessing the intravascular target area of interest and positioning the catheter 210 in the optimal location, the user operates the elongated body actuator 360 positioned within the actuator recess 370 on the handle 230 to expand the catheter 210 and deploy the expandable structure 300. The rearward operation of the elongated body actuator 360 (see FIG. 5) may result in the outer sleeve 470 retracting rearwardly, thereby exposing the expandable structure 300 and permitting the expandable structure 300 to assume a relaxed, expanded state wherein the one or more support arms 400 flare outwardly, as shown in FIG. 16. The location of the expandable structure 300, the support arms 400, the electrodes 410, and the sensors 420 may be facilitated by the imaging apparatus 280 and/or the central imaging apparatus 355, and/or external imaging utilizing the radiopaque markers 270 (shown in FIG. 5).

FIG. 16 shows the expandable structure 300 positioned and deployed in an expanded condition within a curved atherosclerotic portion 700 of the renal artery 80 (similar to the portion 142 shown in FIG. 2) according to one embodiment of the present disclosure. The support arms 400 have expanded outwardly from the longitudinal axis CA, thereby permitting the electrodes 410 and sensors 420 located on the support arms 400 to contact the internal luminal surface 710 of the vessel 700. The luminal surfaces 710a, 710b correspond to raised, irregular inner surfaces of the vessel 700 that have been deformed by a circumferential atherosclerotic plaque 720. The luminal surface 710a covers the thinnest portion of the plaque 720, unlike the luminal surface 710b, which covers a thicker portion of the plaque 720. As shown, the expandable structure 300 has been positioned adjacent to the luminal surface 710a, which is an optimal intravascular position for thermal neuromodulation because of the relatively small intra-to-extravascular distance D6.

An apex of the medial section 550a of first support arm 400a is extended a first distance D4 from the guidewire lumen 510 while permitting an electrode 410a and a sensor 420a positioned thereon to remain in contact with the vessel wall 710a. The first distal tip 730a of the support arm 400a is positioned adjacent to or proximate to the first support arm retainer 540a within the retainer sleeve 530. A second support arm 400b has an apex that is positioned a second distance D5 from to the guidewire lumen 510 while permitting an electrode 410a and a sensor 420b positioned thereon to remain in contact with the vessel wall 710b, wherein the second distance D5 is smaller than the first distance D4. The second distal tip 730b of the second support arm 400b is positioned distally from the retainer 540b within the retainer sleeve 530. As a result, the electrodes 410a, 410b and the sensors 420a, 420b positioned on each of the support arms 400a, 400b remain in contact with the vessel wall 710 despite the disparity between distances D4 and D5.

Thus, as a result of the expandable structure 300 expanding radially outwards, the at least one electrode 410 located on the at least one support arm 400 radially engages the luminal wall 710. Wall-contacting electrodes facilitate more efficient transfer of thermal energy across the vessel wall 710 to the target nerve fibers 120 than electrodes positioned away from the wall 710. At step 626, to aid in registering the electrodes 410 with the circumferential luminal wall 710 of the vessel 700, the user and/or the processor 320 may perform intravascular imaging or external imaging of the distinctively shaped radiopaque markers, such as 600b, of various support arms 400. At step 628, the user and/or the processor 320 may utilize such imaging to determine the circumferential placement of particular electrodes 410 and to refine the treatment plan. Utilizing the intravascular image data provided by the imaging apparatus 280 and/or the central imaging apparatus 355, the user and/or the processor 320 may plan to apply uniform heating of all the electrodes 410 or differential heating by selectively activating or energizing an individual electrode 410 or a selective subset of electrodes 410 with varying amounts of thermal energy, e.g., RF energy, to apply the optimal amount and type of thermal energy to the renal nerves 120 surrounding the vessel 700 to properly denervate the target area.

At step 630, before initializing the application of thermal energy, the user and/or the processor 320 may utilize the electrodes 410, the sensors 420, and/or any auxiliary sensors to sense baseline measurements of various cardiovascular and neurological characteristics of the vessel, including by way of non-limiting example, vessel wall temperature, vessel lumen temperature, the temperature of surrounding non-target tissue, vessel wall impedance and/or conductivity at the target site (i.e., at points of electrode contact with the vessel wall). For example, by emitting a low voltage pulse from the electrodes 410 through the vessel wall and measuring the electrical response, a baseline impedance for the vessel wall at a particular position may be established.

At step 632 of FIG. 15a, the user and/or the processor 320 may utilize such baseline data to refine the treatment plan. For example, utilizing this baseline data, the user and/or the processor 320 may plan to apply uniform heating of all the electrodes 410 or differential heating by selectively activating or energizing an individual electrode 410 or a selective subset of electrodes 410 with varying amounts of thermal energy to apply the optimal amount and type of thermal energy to the renal nerves 120 surrounding the vessel 700 to properly denervate the target area. Throughout the thermal neuromodulation procedure, the baseline measurements may be utilized as a reference against which changes in impedance or conductivity may be compared upon application of thermal energy to the target vessel site.

At step 634 of FIG. 15b, the user and/or the processor 320 may initiate the actual thermal neuromodulation process by applying thermal (i.e., RF) energy to the renal nerves 120 through the electrodes 410. Initially, the thermal field generator 325 generates a thermal electric field, which is selectively transferred to an individual electrode 410 or a selective subset of electrodes 410 on the expandable structure 300. A bipolar electric field may be generated between electrodes 410 positioned on the expandable structure 300, or a monopolar electric field may be delivered between the electrode 410 and the neutral electrode or ground pad 350 (shown in FIG. 1). This thermal energy is transferred from the activated electrodes 410 to the nerves 120 across the vessel wall 710. The electric field thermally modulates the electrical activity along the nerve fibers 120 that control the sympathetic activity of the kidney through the application of heat. This thermal neuromodulation may ablate the nerves 120 or produce non-ablative injury in the nerves 120. Desired neuromodulative effects may include raising the temperature of target nerves 120 over a certain threshold to achieve non-ablative neuromodulation, and raising the temperature of target nerves 120 over an even higher threshold to achieve non-ablative neuromodulation. For example, in some instances, desired neuromodulative effects may occur as a result of raising the temperature of the target nerves to a temperature ranging from about 42 to about 48 degrees Celsius. In most instances, the temperature of the target nerves should not be raised above 62 degrees Celsius to avoid breakdown of the surrounding tissue. These temperature ranges and thresholds are provided for illustrative purposes only, and are not intended to be limiting.

Additionally or alternatively, desired neuromodulative effects may include lowering the temperature of target nerves 120 under a certain threshold to achieve non-ablative neuromodulation, and lowering the temperature of target nerves 120 over an even lower threshold to achieve non-ablative neuromodulation. The electric field may also induce electroporation in the nerve fibers 120.

The non-target tissues surrounding the expandable structure 300 may be protected by focusing the delivery of thermal energy on the target neural fibers 120 such that the intensity of thermal energy affecting the non-target tissues is insufficient to induce serious damage to the non-target tissues. Nevertheless, the surrounding non-target tissues of the vessel wall 710 may also become heated and experience an increase in temperature during delivery of the thermal energy which may damage certain non-target tissues. During the neuromodulation process, the blood flowing through the spaces 580 and passage 590 of the expandable structure may act as a heat sink enabling the conductive and/or convective transfer of heat from the non-target tissue to the blood, thereby protecting the non-target tissue. With blood flowing through the vessel and across the electrodes, more thermal energy may be carried away from the non-target tissues, thereby enabling the use of longer and higher energy neuromodulation treatments. Therefore, the open, basket-like configuration of the expandable structure 300 enables the application of higher energy and longer thermal neuromodulation treatments than would a device that blocked or impeded blood flow.

The user and/or the processor directs the application of thermal energy to target nerves at a specific location for a desired amount of time. In some instances, the desired amount of time may be predetermined by the baseline calculations and/or the patient's underlying vascular pathology, depending upon the condition of the patient's vascular tissue and surrounding tissues. In other instances, the duration of the application of thermal energy to a specific target may vary depending upon imaging results obtained during the procedure. In some instances, a desired neuromodulative effect is attained after application of thermal energy to a target location for about 30 seconds to about 2 minutes. This exemplary duration is provided for illustrative purposes only and is not intended to be limiting.

After applying thermal energy at one target location in the vessel, the user and/or processor may reposition the expandable structure 300 within the lumen and apply thermal energy at another location along the vessel. In some instances, the user and/or processor may reposition the expandable structure 300 by rotating the catheter 210 and/or the expandable structure 300. In some instances, the user and/or processor may reposition the expandable structure 300 by moving the catheter 210 and/or the expandable structure 300 linearly (i.e., proximally or distally) through the lumen of the vessel. The linear distance between two adjacent areas of application may be predetermined by the baseline calculations and/or the patient's underlying vascular pathology, depending upon the condition of the patient's vascular tissue and surrounding tissues. In other instances, the linear distance between two adjacent areas of application may vary depending upon imaging results obtained during the procedure. For example, in some instances, the linear distance between two adjacent areas of application may range from about 1 to 3 mm. In one instance, the linear distance between two adjacent areas of application may be 2 mm. These distances are provided for illustrative purposes only, and are not intended to be limiting.

In some embodiments, the user and/or the processor 320 may direct the application of thermal energy to the plaque to ablate or remodel the plaque and/or reduce the plaque thickness prior to the thermal neuromodulation procedure. Such treatment may be tailored to short term and/or long term increases in lumen diameter and blood flow through the vessel of interest. In some embodiments, remodeling of the atherosclerotic plaque may comprise the use of higher energies to ablate and remove occlusive material from within vessel lumens, and particularly to remove atherosclerotic material from the blood vessel in order to improve blood flow.

At step 636, as shown in FIGS. 15b and 16, as a result of the expandable structure 300 expanding radially outwards, the at least one sensor 420 located on the at least one support arm 400 radially contacts the luminal wall 710, thereby enabling the measurement of the vessel wall temperature. Simultaneously, if provided, the ancillary sensor 575 located proximate to the expandable structure 300 on an exterior surface of the guidewire lumen 510 may measure a characteristic of the environment surrounding the target area, e.g., the blood temperature within the blood vessel, without contacting the vessel wall 710. Both the sensor 420 and the ancillary sensor 575 may send the collected data to the sensor coupler 380 and/or the interface 240 via at least one sensor cable 570, after which the data is transmitted to the controller 310 and the processor 320.

At step 638, the user and/or the processor 320 may control or modulate the thermal neuromodulation by using the measured parameters as feedback. For example, in some embodiments, at least one sensor 420 may be configured as a temperature sensor able to measure the temperature of the vessel wall and/or the non-target tissue. In step 640, if the sensed temperature falls above a therapeutic range indicating a safe range for thermal neuromodulation or the sensed temperature reaches a temperature indicating the desired level of renal nerve injury or ablation, the system 200 may be configured to alert the user and/or the processor to stop the application of thermal energy at step 642. For example, in some instances, desired neuromodulative effects may occur as a result of raising the temperature of the target nerves to a temperature ranging from about 42 to about 48 degrees Celsius. For example, in some embodiments, the sensed vessel wall temperature should not exceed approximately 62 degrees Celsius. These temperature thresholds are provided for illustrative purposes only, and are not intended to be limiting.

At step 644, if the sensed temperature falls within the therapeutic range indicating a safe range for thermal neuromodulation or the sensed temperature has not yet reached a temperature indicating the desired level of renal nerve injury or ablation, the system 200 may be configured to alert the user and/or the processor to continue the application of thermal energy and/or refine the treatment plan at step 646. The potential for undesirably injuring the non-target tissue may be weighed against the expected benefits of thermally neuromodulating the target tissue.

In alternate embodiments, at least one sensor 420 may be configured as an impedance or conductance sensor, obtaining data about the impedance of the vessel wall 710 at any given point. Such sensors may measure the impedance of alternating current (AC) circuits between the electrode 410 and the vessel wall 710, and may include a measurement of both a real portion or magnitude of the impedance, and an imaginary portion or phase angle of the impedance. The impedance magnitude and phase angle generated at an appropriate frequency by the portion of the vessel wall 710 coupled to the electrode may provide a tissue signature. To enhance the accuracy of tissue signature measurements, a plurality of individual measurements may be taken and averaged. By measuring tissue signatures at a plurality of different frequencies within a frequency range, a signature profile for the portion of the vessel wall 710 may be generated. In some embodiments, the various tissue signature measurements about a circumferential portion of the vessel wall 710 may be compared to distinguish between healthy tissue, calcified plaque, fibrous plaque, lipid-rich plaques, untreated tissue, partially treated tissue, fully treated tissue, and the like. The user and/or the processor 320 may use the tissue profiles to determine where in the vessel wall 710 the patient requires more neuromodulation and/or the effectiveness of the applied neuromodulation treatment.

In alternate embodiments, at least one sensor 420 may be configured as a sensor of nerve conductivity/traffic/activity, obtaining data about the neurological activity of the renal nerves 120 overlying the vessel wall at any given point before, during, and/or after the neuromodulation procedure. Such sensors may measure the neurological activity of the renal nerves 120 overlying the vessel wall 710, and may include a measurement of afferent and/or efferent conductivity. In some embodiments, the various neurological conductivity measurements about a circumferential portion of the vessel wall 710 may be compared to distinguish healthy neural tissue from damaged or ablated neural tissue. The user and/or the processor 320 may use the sensed data about neural conductivity/activity/traffic to determine where neural plexus overlying in the vessel wall 710 the patient requires more neuromodulation and/or the effectiveness of the applied neuromodulation treatment. In some embodiments (not pictured in FIG. 15b), the user and/or the processor 320 may use the sensed data about neural conductivity/activity/traffic to determine whether the patient requires more neuromodulation after the other sensed data and/or imaging data suggest that the thermal neuromodulation procedure is complete.

In some embodiments, at least one sensor 420 may be configured as a chemical or hormonal sensor, obtaining data about the sympathetic activity of the patient within the vessel 700. For example, the sensor 420a may monitor a norepinephrine level with the patient's blood, e.g., within the renal vessel 700. Elevated norepinephrine levels may indicate elevated sympathetic activity. If the norepinephrine level rises above a certain threshold, the sensor 420a may monitor renal blood flow and/or renal blood pressure within the renal artery 700. Because sympathetic efferent activation causes renal vasoconstriction and a reduction in renal blood flow, blood flow and/or blood pressure in the renal vessel 700 may indicate the level of renal sympathetic activity. If blood flow to kidneys is decreased and/or renal blood pressure is increased, the sensor 420a may identify an increase in sympathetic activity and send data reflecting this information to the user (via the controller 310) and the processor 320. Once the blood flow and/or blood pressure return to normal, the sensor 420a may switch back to monitoring norepinephrine levels. In alternate embodiments, the expandable structure 300 utilizes a plurality of sensors, e.g., 420a and 420b, to obtain data reflective of changes in renal sympathetic activity.

The user and/or the processor 320 may identify changes in the sympathetic activity level of a patient based on one or more sensed physiological parameters, such as, by way of non-limiting example, blood pressure, blood flow, and/or norepinephrine levels, and control thermal energy delivery to the renal nerves 120 in response to the identified changes. The user and/or the processor 320 may use the sensed physiological parameters to determine when the patient requires more neuromodulation and/or the minimum level of neuromodulation required to maintain renal sympathetic activity below a desired level.

In some embodiments, as shown by steps 636-646 in FIG. 15b, the sensors 420, 575 cooperate with the processor 320 and the electrodes 410 to create a closed feedback loop wherein the processor 320 continuously or intermittently refines the treatment plan and application of thermal energy by directing an individual electrode or a particular combination of electrodes to deliver a particular type, magnitude, and duration of thermal energy depending upon the data received from the sensors 420, 575. Alternatively or additionally, the user may refines the treatment plan and application of thermal energy by directing an individual electrode or a particular combination of electrodes to deliver a particular type, magnitude, and duration of thermal energy with or without depending upon the data received from the sensors 420, 575. In one example, individual electrodes can be actuated to define a generally helical pattern extending around the expandable basket.

In some embodiments, the imaging apparatus 280 and/or central imaging apparatus 355 continue to obtain intravascular image data during the application of thermal energy to the vessel wall 710 to monitor the progress of the renal neuromodulation. In some embodiments, the image data provides evidence of damage to the vessel wall 710, neural injury, and/or neural ablation. At step 648, the user and/or processor may direct the imaging apparatus 280 and/or central imaging apparatus 355 obtain intravascular image data of the vessel wall adjacent the target nerves after the application of thermal energy to the vessel wall 710. At step 650, the user and/or the processor 320 may utilize such data to determine whether the desired level of thermal injury has been achieved. At step 652, if the imaging data leads to an assessment that the desired level of thermal injury and/or neuromodulation has been achieved, the user and/or the processor 320 may stop the application of thermal energy. If, at step 654, user and/or the processor 320 use the imaging data to determine that the desired level of thermal injury and/or neuromodulation has not been achieved, the user and/or the processor 320 may continue the application of thermal energy and/or refine the treatment plan.

Steps 636-654 of FIG. 15b illustrate how the thermal neuromodulation process may be monitored and controlled by acquiring data from the imaging devices and the sensors along the vessel wall 710 in the region of treatment, and limiting the power and/or duration of the application of thermal energy to the vessel wall 710 in response to that data. For example, in response to the data collected by the imaging devices and the sensors, the user or program algorithms from the processor 320 may selectively direct individual electrodes 410 or combinations of electrodes 410 to apply thermal energy to the vessel wall 710 while other electrodes remain inactive. In addition, the user or program algorithms from the processor 320 may selectively direct individual sensors 420, 575 or combinations of particular sensors 420, 575 to obtain measurements while other sensors remain inactive.

In the course of the neuromodulation process and data collection, the distal portion 260 of the body 220 may be retracted proximally or advanced distally within the vessel 700, while the expandable structure 300 is in an expanded condition, in order to determine a gradient of measurements over a longitudinal length of the vessel. For example, the user may advance and/or retract the expandable structure 300 in 2 millimeter increments to apply thermal energy at various positions within a target vessel. Alternatively, the expandable structure 300 may be repeatedly contracted or unexpanded, and the catheter 210 may be axially moved to reposition the expandable structure 300, with subsequent expansion of the expandable structure 300 at each of a plurality of treatment locations along the vessel 700.

Once the neuromodulation process is initially determined to be complete (for example, at step 652), the user may obtain a final set of intravascular images with the imaging apparatus 280 and/or the central imaging apparatus 355 (for example, at step 648) to examine the condition of the vessel wall 710 as well as evaluate the efficacy of the applied neuromodulation treatment on the renal nerves 120. At step 656, after determining that the neuromodulation process is complete based on the intravascular image data, the user may stop the application of thermal energy and, at step 658, begin the process of removing the thermal basket catheter 210 from the target vessel and the patient's body. Initially, the user may return the elongated body actuator 360 located on the handle 230 to a non-deployed position within the actuator recess 370 (shown in FIG. 5). As a result, the outer sleeve 470 may be advanced towards the distal portion 260, as shown in FIGS. 9 and 11, thereby allowing the body 220 to assume an unexpanded condition. While advancing towards the distal portion 260, an inner wall of the outer sleeve 470 engages and compresses the expandable structure 300 inwardly, thereby permitting the expandable structure 300 to be received within the sleeve lumen 480 and returning the expandable structure 300 to a non-deployed, unexpanded configuration, as illustrated in FIG. 9. Prior to removing the catheter 210 from the blood vessel 700, the user may delivery a therapeutic agent to an area of interest with the catheter 700 through the guidewire port 450, for example. Thereafter, the catheter 210 and the guidewire 460 may be removed from the patient and the entry incisions may be closed.

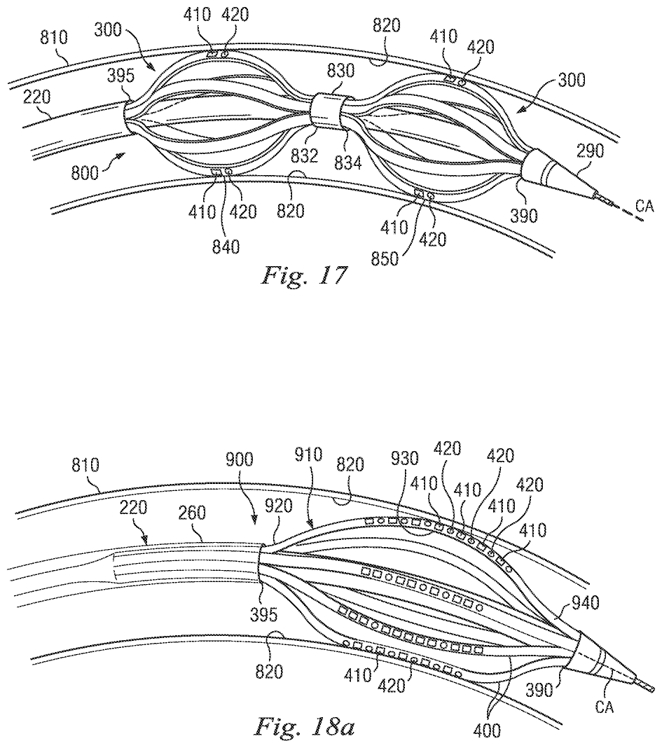

FIG. 17 shows a thermal basket catheter 800 including at least two expandable structures 300 positioned and deployed in an expanded condition within a curved portion 810 of the renal artery 80 (similar to the portion 141 shown in FIG. 2) according to one embodiment of the present disclosure. The thermal basket catheter 800 is substantially identical to the thermal basket catheter 210 except for the differences noted herein. The support arms 400 of each expandable structure 300 have expanded outwardly from the longitudinal axis CA, thereby permitting the electrodes 410 and sensors 420 located on the support arms 400 to contact an internal luminal surface 820 of the vessel 810. The thermal basket catheter 800 may include at least one central support member 830, which is shaped and configured as a hollow tubular portion of the catheter body 220. In the illustrated embodiment, the central support member 830 includes a proximal connection section 832, which connects to the proximal connection part 395 when the catheter 800 is in an unexpanded condition (not shown), and a distal connection section 834, which connects to the distal connection part 390 when the catheter 800 is in an unexpanded condition. Using a thermal basket catheter including multiple expandable structures 300 allows the user to simultaneously apply thermal energy to multiple positions spaced longitudinally along the vessel wall, thereby potentially shortening the duration of the thermal neuromodulation procedure. For example, in the pictured embodiment, the expandable structures 300 may simultaneously apply thermal energy to the vessel wall at a circumferential position 840 and a circumferential position 850, which are spaced longitudinally from each other along the vessel wall of vessel 810.

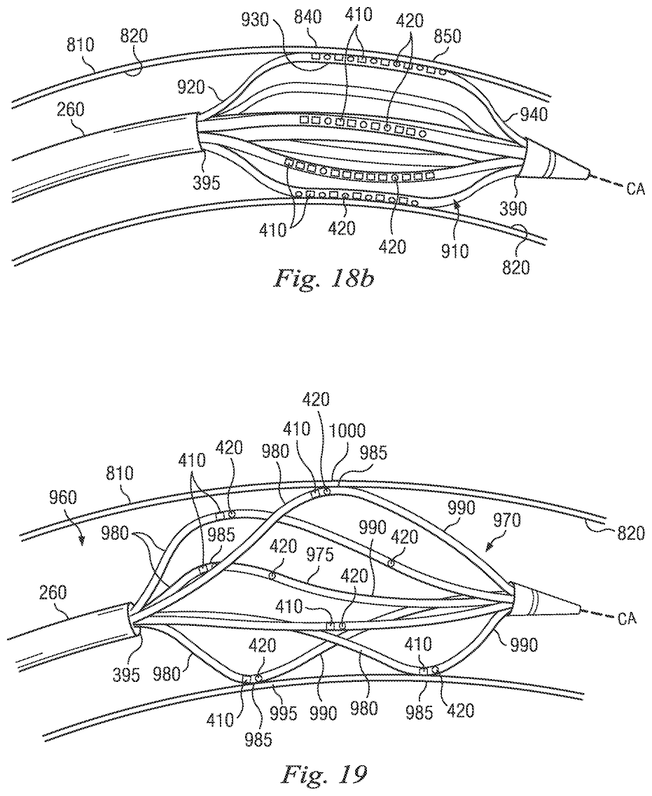

FIGS. 18a and 18b show a thermal basket catheter 900 including an elongated expandable structure 910 positioned within a curved portion 810 of the renal artery 80 (similar to the portion 141 shown in FIG. 2) according to one embodiment of the present disclosure. FIG. 18a illustrates the elongated expandable structure 910 in a partially expanded condition emerging from the proximal connection part 395 of the distal portion 260. The thermal basket catheter 900 is substantially identical to the thermal basket catheter 210 except for the differences noted herein. The expandable structure 910 is shaped and configured as an elongated basket comprising support arms 400 that include proximal parts 920, intermediate parts 930, and distal parts 940. Each intermediate part 930 is shaped and configured as a flattened, elongated section configured to contact an internal luminal surface 820 of the vessel 810 along the length of the intermediate part 930. Each proximal part 920 and distal part 940 is shaped and configured to slope from the intermediate part 930 toward the longitudinal axis CA of the catheter 900.

The support arms 400 of the expandable structure 910 include multiple electrodes 410 and sensors 420, at least some of which are positioned along the intermediate parts 930 of the arms 400. In some embodiments, the majority of electrodes 410 and sensors 420 of the expandable structure 910 are clustered on the intermediate parts 930 of the support arms 400. In FIG. 18a, the support arms 400 of the expandable structure 910 have partially expanded outwardly from the longitudinal axis CA, thereby permitting at least some of the electrodes 410 and the sensors 420 located on the support arms 400 to contact the internal luminal surface 820 of the vessel 810.

FIG. 18b illustrates the elongated expandable structure 910 in an expanded condition after emerging from the proximal connection part 395 of the distal portion 260. In the pictured embodiment, the intermediate parts 930 of the support arms 400 of the expandable structure 910 have expanded outwardly from the longitudinal axis CA, thereby permitting a majority of the electrodes 410 and the sensors 420 located on the support arms 400 to contact the internal luminal surface 820 of the vessel 810. Using a thermal basket catheter including an elongated expandable structure allows the user to simultaneously apply thermal energy to multiple positions spaced longitudinally along the vessel wall, thereby potentially shortening the duration of the thermal neuromodulation procedure. For example, in the pictured embodiment, the expandable structure 910 may simultaneously apply thermal energy to the vessel wall at a circumferential position 840 and a circumferential position 850, which are spaced longitudinally from each other along the vessel wall of vessel 810.

FIG. 19 shows a thermal basket catheter 960 including a helical expandable structure 970 positioned within a curved portion 810 of the renal artery 80 (similar to the portion 141 shown in FIG. 2) according to one embodiment of the present disclosure. FIG. 19 illustrates the elongated expandable structure 960 in an expanded condition after emerging from the proximal connection part 395 of the distal portion 260. The thermal basket catheter 970 is substantially identical to the thermal basket catheter 210 except for the differences noted herein. The expandable structure 970 is shaped and configured as an elongated basket comprising support arms 975 that include proximal parts 980, intermediate parts 985, and distal parts 990.

The support arms 975 of the expandable structure 970 include multiple electrodes 410 and sensors 420, at least some of which are positioned along the intermediate parts 985 of the arms 975. In the pictured embodiment, the majority of electrodes 410 and sensors 420 of the expandable structure 960 are clustered on the intermediate parts 985 of the support arms 400. Each arm 975 is shaped and configured to flex at the intermediate part 985, thereby enabling the electrode 420 and/or the sensor 410 to contact an internal luminal surface 820 of the vessel 810. Each proximal part 980 and distal part 990 is shaped and configured to slope from the intermediate part 985 toward the longitudinal axis CA of the catheter 960. The intermediate parts 985, or apex, of each arm 975 in the expanded configuration are staggered longitudinally such that in the expanded condition the intermediate parts align in a generally helical pattern circumferentially extending around the longitudinal axis. In the illustrated embodiments, many arms 975 have a short portion and a long portion that defines the intermediate part 985 therebetween.

In the pictured embodiment, the intermediate parts 985 of the support arms 975 of the helical expandable structure 970 have expanded outwardly from the longitudinal axis CA, thereby permitting a majority of the electrodes 410 and the sensors 420 located on the support arms 400 to contact the internal luminal surface 820 at different linearly-spaced locations along the length of the vessel 810. Such a configuration allows the expandable structure 970 to contact and apply thermal energy to various, linearly-spaced areas along the intraluminal surface, thereby reducing or preventing circumferential thermal injury to a focal, ring-like area of the vessel tissue. In some instances, the expandable structure 970 allows the user and/or processor to apply an energy in a helical or spiral pattern to the intraluminal surface 82-820. Using a thermal basket catheter including a helical expandable structure allows the user to simultaneously apply thermal energy to multiple positions spaced longitudinally along the vessel wall, thereby potentially shortening the duration of the thermal neuromodulation procedure. For example, in the pictured embodiment, the expandable structure 970 may simultaneously apply thermal energy to the vessel wall at a circumferential position 995 and a circumferential position 1000, which are spaced longitudinally from each other along the vessel wall of vessel 810.

It should be appreciated that while several of the exemplary embodiments herein are described in terms of an ultrasonic device, or more particularly the use of IVUS data (or a transformation thereof) to render images of a vascular object, the present disclosure is not so limited. Thus, for example, an imaging device using backscattered data (or a transformation thereof) based on ultrasound waves or even electromagnetic radiation (e.g., light waves in non-visible ranges such as Optical Coherence Tomography, X-Ray CT, etc.) to render images of any tissue type or composition (not limited to vasculature, but including other human as well as non-human structures) is within the spirit and scope of the present disclosure.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. For example, the thermal basket catheter may be utilized anywhere with a patient's vasculature, both arterial and venous, having an indication for thermal neuromodulation. It is understood that such variations may be made to the foregoing without departing from the scope of the present disclosure. Accordingly, it is appropriate that the appended claims be construed broadly and in a manner consistent with the present disclosure.

We claim:

1. An apparatus for intravascular thermal neuromodulation, comprising:
a processor;
a catheter in communication with the processor, wherein the catheter comprises:
an elongate body configured to be inserted into a blood vessel, wherein the elongate body includes a proximal portion and a distal portion, and wherein the distal portion includes a distal tip; and
an expandable structure comprising a plurality of support arms, wherein each of the plurality of support arms comprises a proximal section, an intermediate section, and a distal section; and
an actuator configured to deploy the expandable structure through a plurality of expansion states with different arrangements of the expandable structure, the elongate body, and the distal tip,
wherein the intermediate section of a support arm of the plurality of support arms comprises a plurality of electrodes longitudinally spaced from one another along the intermediate section and configured to deliver energy to a wall of the blood vessel,
wherein the plurality of expansion states comprises:
a first expansion state in which the distal portion and the distal tip are in contact with each other and the expandable structure is disposed within the distal portion; and
a second expansion state in which:
the distal portion and the distal tip are spaced apart from each other; and
all of the plurality of electrodes simultaneously contact the wall, and
wherein, with the expandable structure deployed in the second expansion state, the processor is configured to selectively control the plurality of electrodes to deliver the energy to:
a length of the wall spanning the entire intermediate section via all of the plurality of electrodes, wherein opposite ends of the length are longitudinally spaced from one another; or
a spot of the wall spanning only a portion of the intermediate section via only a subset of the plurality of electrodes.

2. The apparatus of claim 1, wherein the intermediate section comprises a shape configured for contact with the wall, wherein the shape of the intermediate section is different than a shape of the proximal section and a shape of the distal section.

3. The apparatus of claim 2, wherein the shape comprises a flattened shape.

4. The apparatus of claim 1, wherein the spot of the wall is located between the opposite ends of the length.

5. The apparatus of claim 1, wherein the support arm of the plurality of support arms comprises a plurality of sensors different than the plurality of electrodes.

6. The apparatus of claim 5, wherein the plurality of sensors are longitudinally spaced from one another along the intermediate section.

7. The apparatus of claim 5, wherein the plurality of sensors are interleaved with the plurality of electrodes along the intermediate section such that the plurality of electrodes are longitudinally spaced from one another along the intermediate section by the plurality of sensors.

8. The apparatus of claim 5, wherein the plurality of sensors comprises at least one of an ultrasonic sensor, a flow sensor, a thermal sensor, a blood temperature sensor, an electrical contact sensor, a conductivity sensor, an impedance sensor, an electromagnetic detector, a pressure sensor, a chemical or hormonal sensor, a pH sensor, an infrared sensor, or a contact pressure sensor configured to detect a pressure applied by an electrode of the plurality of electrodes at the wall.

9. The apparatus of claim 5, wherein the plurality of sensors is configured to:
obtain sensor data representative of the blood vessel; and
transmit the sensor data to the processor.

10. The apparatus of claim 9, wherein the processor is further configured to selectively control the plurality of electrodes to deliver the energy based on the sensor data.

11. The apparatus of claim 1, wherein the elongate body further comprises an intravascular ultrasound (IVUS) imaging apparatus configured to obtain images of the wall.

12. The apparatus of claim 11, wherein the IVUS imaging apparatus is positioned within the expandable structure.

13. The apparatus of claim 1, wherein the processor is configured to register the plurality of electrodes at a circumferential position of the expandable structure with a corresponding circumferential location of the blood vessel based on an intravascular image.

14. The apparatus of claim 1, wherein the plurality of electrodes is configured to deliver the energy through the wall to a renal nerve.
